SEQUENCE LISTING (96 sequences)

SEQ ID NO: 1 - DNA, 21 bp, Artificial Sequence
  Synthesized primer
agattcgggatatgctgttgg

SEQ ID NO: 2 - DNA, 21 bp, Artificial Sequence
  Synthesized primer
aaagcctggaagaaggaggtc

SEQ ID NO: 3 - DNA, 23 bp, Artificial Sequence
  Synthesized primer
agaagagcaaaccaccttcagag

SEQ ID NO: 4 - DNA, 22 bp, Artificial Sequence
  Synthesized primer
gtcacagtaggccttgatctcc

SEQ ID NO: 5 - DNA, 26 bp, Artificial Sequence
  Synthesized primer
gatgtggaacccataactggattcac

SEQ ID NO: 6 - DNA, 26 bp, Artificial Sequence
  Synthesized primer
ggtcccagtctcatttagccacagta

SEQ ID NO: 7 - DNA, 20 bp, Artificial Sequence
  Synthesized primer
ggagccaagaactcatcctg

SEQ ID NO: 8 - DNA, 20 bp, Artificial Sequence
  Synthesized primer
tctggcctgctccttaggta

SEQ ID NO: 9 - DNA, 20 bp, Artificial Sequence
  Synthesized primer
cctgtcccctatcgacatgg

SEQ ID NO: 10 - DNA, 19 bp, Artificial Sequence
  Synthesized primer
cttttccggcacttggagg

SEQ ID NO: 11 - DNA, 21 bp, Artificial Sequence
  Synthesized primer
tgggagtgttgtgaagagtga

SEQ ID NO: 12 - DNA, 21 bp, Artificial Sequence
  Synthesized primer
gcagtgaagccgatgaagaac

SEQ ID NO: 13 - DNA, 20 bp, Artificial Sequence
  Synthesized primer
cccatgtgctcctaccagat

SEQ ID NO: 14 - DNA, 20 bp, Artificial Sequence
  Synthesized primer
ccttgaagaagcgacctttg

SEQ ID NO: 15 - DNA, 20 bp, Artificial Sequence
  Synthesized primer
tccatcagggtatcctctcc

SEQ ID NO: 16 - DNA, 20 bp, Artificial Sequence
  Synthesized primer
ggaggcaagcataagactgg

SEQ ID NO: 17 - DNA, 19 bp, Artificial Sequence
  Synthesized primer
actgccactgcaggatgag

SEQ ID NO: 18 - DNA, 20 bp, Artificial Sequence
  Synthesized primer
cacagcctcagcatcttcaa

SEQ ID NO: 19 - DNA, 20 bp, Artificial Sequence
  Synthesized primer
gggagcttgtgttcctcatc

SEQ ID NO: 20 - DNA, 20 bp, Artificial Sequence
  Synthesized primer
atctccatccaggcactctg

SEQ ID NO: 21 - DNA, 20 bp, Artificial Sequence
  Synthesized primer
gatgagcctcctccagagtg

SEQ ID NO: 22 - DNA, 20 bp, Artificial Sequence
  Synthesized primer
tgcacagcttccacatcttc

SEQ ID NO: 23 - DNA, 24 bp, Artificial Sequence
  Synthesized primer
tcctcattccaagaaactctgtcc

SEQ ID NO: 24 - DNA, 22 bp, Artificial Sequence
  Synthesized primer
ggtaagagtggtcttctgtccc

SEQ ID NO: 25 - DNA, 24 bp, Artificial Sequence
  Synthesized primer
ggaggctataaggtacgaaaccag

SEQ ID NO: 26 - DNA, 22 bp, Artificial Sequence
  Synthesized primer
gacccgtattcattctccacca

SEQ ID NO: 27 - DNA, 20 bp, Artificial Sequence
  Synthesized primer
gctctgatgaccgaactcaa

SEQ ID NO: 28 - DNA, 20 bp, Artificial Sequence
  Synthesized primer
attccacgatcaccatcaga

SEQ ID NO: 29 - DNA, 20 bp, Artificial Sequence
  Synthesized primer
actgggcaagtcctttgaga

SEQ ID NO: 30 - DNA, 21 bp, Artificial Sequence
  Synthesized primer
gtctaagccaaacacctgggc

SEQ ID NO: 31 - DNA, 21 bp, Artificial Sequence
  Synthesized primer
cttctttgagattggccctgg

SEQ ID NO: 32 - DNA, 23 bp, Artificial Sequence
  Synthesized primer
aggtgaagatgaagaagccaagc

SEQ ID NO: 33 - DNA, 20 bp, Artificial Sequence
  Synthesized primer
tccatgtgaccatgaggaaa

SEQ ID NO: 34 - DNA, 20 bp, Artificial Sequence
  Synthesized primer
cttccacgttgctgacttga

SEQ ID NO: 35 - DNA, 24 bp, Artificial Sequence
  Synthesized primer
gtagcttatgaatgtgtgcaactc

SEQ ID NO: 36 - DNA, 24 bp, Artificial Sequence
  Synthesized primer
gtcttgcgatcagctctttcatta

SEQ ID NO: 37 - DNA, 25 bp, Artificial Sequence
  Synthesized primer
tgaactcgcccgtcaggctgttgag

SEQ ID NO: 38 - DNA, 28 bp, Artificial Sequence
  Synthesized primer
gatgtcatccagactggggtggacaacc

SEQ ID NO: 39 - DNA, 20 bp, Artificial Sequence
  Synthesized primer
gtagcggagactcggaattg

SEQ ID NO: 40 - DNA, 20 bp, Artificial Sequence
  Synthesized primer
accttctagtccagggctgc

SEQ ID NO: 41 - DNA, 20 bp, Artificial Sequence
  Synthesized primer
gatgcagctgaagggaaaga

SEQ ID NO: 42 - DNA, 20 bp, Artificial Sequence
  Synthesized primer
gtcacctgcctgttcctttg

SEQ ID NO: 43 - DNA, 20 bp, Artificial Sequence
  Synthesized primer
tccatcagccatttcaacaa

SEQ ID NO: 44 - DNA, 20 bp, Artificial Sequence
  Synthesized primer
gttacagagccgaggtggag

SEQ ID NO: 45 - DNA, 19 bp, Artificial Sequence
  Synthesized primer
cttcgcgtaaccgaggatt

SEQ ID NO: 46 - DNA, 22 bp, Artificial Sequence
  Synthesized primer
gggactagtgatcagttcatgg

SEQ ID NO: 47 - DNA, 20 bp, Artificial Sequence
  Synthesized primer
ccaagggcctgaatgaggag

SEQ ID NO: 48 - DNA, 20 bp, Artificial Sequence
  Synthesized primer
gcaaaggctccaggtctgag

SEQ ID NO: 49 - DNA, 20 bp, Artificial Sequence
  Synthesized primer
acaagctgcgggtgaagagc

SEQ ID NO: 50 - DNA, 20 bp, Artificial Sequence
  Synthesized primer
caggacagtgacaaagaacg

SEQ ID NO: 51 - DNA, 20 bp, Artificial Sequence
  Synthesized primer
aagcgaagagtaaggctgtc

SEQ ID NO: 52 - DNA, 20 bp, Artificial Sequence
  Synthesized primer
gtgattgcttgcaaaggaac

SEQ ID NO: 53 - DNA, 20 bp, Artificial Sequence
  Synthesized primer
ccaagtgcaggaaagtgacc

SEQ ID NO: 54 - DNA, 20 bp, Artificial Sequence
  Synthesized primer
aggaagagactgacgagctc

SEQ ID NO: 55 - DNA, 26 bp, Artificial Sequence
  Synthesized primer
ctgacgaaggccactttgcacctctg

SEQ ID NO: 56 - DNA, 28 bp, Artificial Sequence
  Synthesized primer
gcacaagatcccggtcaagtacctggag

SEQ ID NO: 57 - DNA, 20 bp, Artificial Sequence
  Synthesized primer
ctggtctttcagccaccatt

SEQ ID NO: 58 - DNA, 20 bp, Artificial Sequence
  Synthesized primer
ccttcattggatcctccatc

SEQ ID NO: 59 - DNA, 20 bp, Artificial Sequence
  Synthesized primer
cgctgcaggagaggtttatc

SEQ ID NO: 60 - DNA, 20 bp, Artificial Sequence
  Synthesized primer
cctgcatctgctgtgaggta

SEQ ID NO: 61 - DNA, 19 bp, Artificial Sequence
  Synthesized primer
ggtgttgatgttcccgcct

SEQ ID NO: 62 - DNA, 21 bp, Artificial Sequence
  Synthesized primer
tcagtgatgaggaccagagcg

SEQ ID NO: 63 - DNA, 19 bp, Artificial Sequence
  Synthesized primer
ccactccatccgctccttt

SEQ ID NO: 64 - DNA, 19 bp, Artificial Sequence
  Synthesized primer
aagtccagctcagccccat

SEQ ID NO: 65 - DNA, 23 bp, Artificial Sequence
  Synthesized primer
aaaggacaggatggaaggaatca

SEQ ID NO: 66 - DNA, 24 bp, Artificial Sequence
  Synthesized primer
attaactggcagagtggcaggtaa

SEQ ID NO: 67 - DNA, 23 bp, Artificial Sequence
  Synthesized primer
aagtgtggaactctctggaactg

SEQ ID NO: 68 - DNA, 23 bp, Artificial Sequence
  Synthesized primer
gggttatcttggttggctttatg

SEQ ID NO: 69 - DNA, 20 bp, Artificial Sequence
  Synthesized primer
tggagactgctctggaaggt

SEQ ID NO: 70 - DNA, 20 bp, Artificial Sequence
  Synthesized primer
tgctgctgtcctcaaatacg

SEQ ID NO: 71 - DNA, 23 bp, Artificial Sequence
  Synthesized primer
cctgaacatcgagtgtcgaatat

SEQ ID NO: 72 - DNA, 25 bp, Artificial Sequence
  Synthesized primer
ggttcttcttctgaatcttgcagct

SEQ ID NO: 73 - DNA, 23 bp, Artificial Sequence
  Synthesized primer
ctcttcatcgcggccatcattct

SEQ ID NO: 74 - DNA, 23 bp, Artificial Sequence
  Synthesized primer
tctgccatcttctgcagcagctt

SEQ ID NO: 75 - DNA, 24 bp, Artificial Sequence
  Synthesized primer
atgggtgaaactctgggagattct

SEQ ID NO: 76 - DNA, 25 bp, Artificial Sequence
  Synthesized primer
cttggagcttcaggtcatatttgta

SEQ ID NO: 77 - DNA, 20 bp, Artificial Sequence
  Synthesized primer
ggacctatggtgttggatgc

SEQ ID NO: 78 - DNA, 20 bp, Artificial Sequence
  Synthesized primer
gtgtgcacgccagagtattg

SEQ ID NO: 79 - DNA, 20 bp, Artificial Sequence
  Synthesized primer
tgtcacctcagccagcatag

SEQ ID NO: 80 - DNA, 20 bp, Artificial Sequence
  Synthesized primer
cgccgtaagtagtcctcctg

SEQ ID NO: 81 - DNA, 19 bp, Artificial Sequence
  Synthesized primer
tggcaggtagcaccacagg

SEQ ID NO: 82 - DNA, 22 bp, Artificial Sequence
  Synthesized primer
catctggtcttgcagcaactct

SEQ ID NO: 83 - DNA, 25 bp, Artificial Sequence
  Synthesized primer
actccagcgtcgccatcaggattct

SEQ ID NO: 84 - DNA, 25 bp, Artificial Sequence
  Synthesized primer
taaacaggtgagactccagcaactt

SEQ ID NO: 85 - DNA, 20 bp, Artificial Sequence
  Synthesized primer
aacgatgaagccctggagtg

SEQ ID NO: 86 - DNA, 19 bp, Artificial Sequence
  Synthesized primer
tgagaggtctggttcccga

SEQ ID NO: 87 - DNA, 20 bp, Artificial Sequence
  Synthesized primer
aacgatgaagccctggagtg

SEQ ID NO: 88 - DNA, 21 bp, Artificial Sequence
  Synthesized primer
gacaaacaaatgctttctccg

SEQ ID NO: 89 - DNA, 20 bp, Artificial Sequence
  Synthesized primer
aacgatgaagccctggagtg

SEQ ID NO: 90 - DNA, 20 bp, Artificial Sequence
  Synthesized primer
aacaaggctcacagtgaacg

SEQ ID NO: 91 - DNA, 20 bp, Artificial Sequence
  Synthesized primer
agccaccagaagaaagtggt

SEQ ID NO: 92 - DNA, 20 bp, Artificial Sequence
  Synthesized primer
gctgggcactagttgtttga

SEQ ID NO: 93 - DNA, 50 bp, Artificial Sequence
  Synthesized oligonucleotide
caccgcgtcagctttctgtgattccccaaggaatcacagaaagctgacgc

SEQ ID NO: 94 - DNA, 50 bp, Artificial Sequence
  Synthesized oligonucleotide
aaaagcgtcagctttctgtgattccttcgggaatcacagaaagctgacgc

SEQ ID NO: 95 - DNA, 50 bp, Artificial Sequence
  Synthesized oligonucleotide
caccgggtctatctctctgggattgcgaacaatcccagagagatagaccc

SEQ ID NO: 96 - DNA, 50 bp, Artificial Sequence
  Synthesized oligonucleotide
aaaagggtctatctctctgggattgttcgcaatcccagagagatagaccc

(19) United States
(12) Patent Application Publication
(10) Pub. No.: US 2014/0147434 A1
(43) Pub. Date: May 29, 2014

(54) NCoR1 is a Physiological Modulator of Muscle Mass and Oxidative Function

(71) Applicants: Johan Auwerx, Buchillon (CH); Hiroyasu Yamamoto, Suita City (JP); Laurent Mouchiroud, Lausanne (CH)

(72) Inventors: Johan Auwerx, Buchillon (CH); Hiroyasu Yamamoto, Suita City (JP); Laurent Mouchiroud, Lausanne (CH)

(73) Assignee: ECOLE POLYTECHNIQUE FEDERALE DE LAUSANNE (EPFL), Lausanne (CH)

(21) Appl. No.: 14/116,095

(22) PCT Filed: May 7, 2012
(86) PCT No.: PCT/IB2012/001044

§ 371 Date: Feb. 10, 2014

Related U.S. Application Data
(60) Provisional application No. 61/483,326, filed on May 6, 2011.

(51) Int. Cl. A61K 39/395; A61K 31/713

(52) U.S. Cl. CPC A61K 39/3955; A61K 31/713
USPC 424/130.1; 514/44 A; 435/375

(57) ABSTRACT

The present invention provides methods of increasing muscle mass and muscle mitochondrial oxidative metabolism. Additionally, the invention provides treating various disorders associated with mitochondrial dysfunction, including but not limited to metabolic disorders, muscular dystrophy disorders, neurodegenerative diseases, chronic inflammatory diseases, and diseases of aging.

### RELATED APPLICATIONS

This application claims the benefit of provisional application U.S. Ser. No. 61/483,326, filed May 6, 2011 the contents which is herein incorporated by reference in its entirety.

### FIELD OF THE INVENTION

The present invention relates generally to methods of increasing muscle mass or muscle oxidative metabolism as well as for the treatment of muscular dystrophy disorder and various mitochondrial disorders, including metabolic disorders and genetic mitochondrial disease.

### BACKGROUND OF THE INVENTION

Transcription factors are key mediators in homeostatic circuits, as they process environmental signals into transcriptional changes (Desvergne et al., 2006; Francis et al., 2003). Transcriptional coregulators have recently emerged as equally important modulators of such adaptive transcriptional responses. The fact that the activity of coactivators and corepressors is tightly regulated through the spatial and temporal control of their expression and activity levels opens hence another avenue to adapt transcription to environmental cues (Feige and Auwerx, 2007; Rosenfeld et al., 2006; Smith and O′Malley, 2004; Spiegelman and Heinrich, 2004). Interestingly, many of these coregulators do not operate in isolation, but are part of large multi-protein complexes, that integrate complex signaling pathways. The convergence of an elaborate coregulator network on the peroxisome proliferator-activated receptor (PPAR) coactivator α (PGC)-1α illustrates this principle well, as its activity depends on several other coregulators, including the steroid receptor coactivators, NR interacting protein 1 or RIP140, CREB binding protein, p300, protein arginine methyltransferase 1, general control of amino acid synthesis 5, and SIRT1 (Fernandez-Marcos and Auwerx, 2011; Handschin and Spiegelman, 2006).

The corepressor (NCoR1) and the silencing mediator for retinoid and thyroid hormone receptor (SMRT or NCoR2) are also acting as cofactor scaffolding platforms. NCoR1 and SMRT hardwire corepressor pathways that incorporate several deacetylases [including class I (HDAC3), class II (HDAC4, 5, 7, and 9) and class III (SIRT1) HDACs], transducin beta-like 1 (TBL1) and TBLR1, two highly related F box/WD40-containing factors, and the G-protein-pathway suppressor 2 [reviewed in (Perissi et al., 2010)]. Since germline NCoR1^{-/-} and SMRT mice are embryonically lethal (Jepsen et al., 2000; Jepsen et al., 2007), information on the role of these proteins in adult physiology is limited. Studies of mice with mutations in the NR interaction domains (RIDs) 1 and 2 of SMRT (SMRTmRID), which solely disrupts its interaction with NRs, indicated that lethality of SMRT^{-/-} mice is caused by non-NR transcription factors (Nofsinger et al., 2008). Work in 3T3-L1 cells in which NCoR1 or SMRT expression was reduced by RNA interference, demonstrated that they repress adipogenesis by inhibiting PPARγ (Yu et al., 2005). In line with this, adipogenesis was enhanced in mouse embryonic fibroblasts (MEFs) from SMRTmRID mice (Nofsinger et al., 2008). Interestingly, SIRT1 is also part of the NCoR1/SMRT complex and contributes to the inhibition of PPARγ (Picard et al., 2004).

Contrary to adipose tissue, the function of NCoR1/SMRT in skeletal muscle has not yet been established. Thus a need exists to elucidate the function of NCoR1 in skeletal muscle.

### SUMMARY OF THE INVENTION

The invention features methods of increasing muscle mass or muscle mitochondrial oxidative metabolism by administering to subject in need thereof one or more compounds that decrease muscle-specific nuclear receptor corepressor 1 (NCoR1) expression or activity.

Also included in the invention are methods of increasing mitochondrial number and function in a cell by contacting a cell with one or more compounds that decrease muscle-specific nuclear receptor corepressor 1 (NCoR1) expression or activity. The cell is a muscle cell or an adipocyte.

In another aspect the invention provides a method of treating a disorder associated with mitochondrial dysfunction or a muscular dystrophy disorder by administering to a subject in need thereof one or more compounds that decrease muscle-specific nuclear receptor corepressor 1 (NCoR1) expression or activity.

A disorder associated with mitochondrial dysfunction is a metabolic disorder, a neurodegenerative disease, a chronic inflammatory disease, or an aging related disorder. For example, the metabolic disorder is obesity or type II diabetes. The muscular dystrophy disorder is an inherited muscular dystrophy disorder or an acquired muscular dystrophy disorder.

The compound is a compound is a NCoR1 antibody or a nucleic acid that inhibits NCoR1 expression or activity. The compound inhibits or dissociates a NCoR1-histone deacetylases (HDAC) complex. In one aspect the compound is an HDAC inhibitor.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1: Generation and Validation of Mice with a Targeted Mutation of NCoR1 in Muscle.

(A) Gene targeting and conditional deletion of exon 11 of the NCoR1 gene. Maps of the NCoR1 genomic locus, the floxed allele with the neomycin cassette (+neo) (target allele) and without the neomycin cassette (−neo) (conditional allele). The white and black arrows indicate the primers used for PCR assessment of recombination. Positions of the exons are indicated. (B) LoxP sites (L2) in the NCoR1 locus were determined by PCR amplification on the genomic DNA from HSAcre^{0/0}/NCoR1^{WT/WT}, HSAcre^{Tg/0}/NCoR1^{WT/WT}, HSAcre^{0/0}/NCoR1^{L2/WT}, HSAcre^{Tg/0}/NCoR1^{L2/WT}, HSAcre^{0/0}/NCoR1^{L2/L2}, and HSAcre^{Tg/0}/NCoR1^{L2/L2} mice using specific primer sets at the 5 ‘-end and 3’-end of exon 11 (FIG. 2A). The presence of the CRE gene was assessed using specific primers for Cre recombinase, with myogenin as a control. (C) Excision of NCoR1 locus by Cre-mediated DNA recombination is confined to muscle. Tissue specificity of recombination was assessed by PCR analysis on genomic DNA isolated from soleus muscle (S), gastrocnemius muscle (G), liver (L) tail (T) of mice described in panel A. Sequence of primer sets is available upon request.

FIG. 2: Validation and Metabolic Phenotypes of NCoR1^{skm-/-} mice.

(A) mRNA levels of NCoR1 and Smrt in different tissues were determined by qRT-PCR. Values were normalized to 36B4. (n=8-10/group). (B) Biochemical analysis of the plasma from NCoR1^{skm+/+} and ^{skm-/-} mice after 6 hr fasting (n=8) either on chow diet (CD) (top) or HFD (bottom). (C) Circadian activity, measured as the total locomotor activity, and energy expenditure was evaluated by the measurement of oxygen consumption (VO₂), and by the calculation of the respiratory exchange ratio (RER) over a 24 hr period after 12 wks of HFD. The bar graphs represent the average for each group (n=12). (D) Body temperature was measured for 7 hr in mice exposed to 4° C. after 18-wks of HFD (n=7, 8). (E and F) Exercise experiments, measuring running time and distance till exhaustion (endurance exercise) (E) and the increment of VO₂ₘₐₓ, during exercise, and RER levels at basal and VO₂ₘₐₓ condition (F) were performed after 14- and 17-wk HFD. Data are expressed as mean±SEM.

FIG. 3: Metabolic Phenotypes of the NCoR1^{skm+/+} and ^{skm-/-} mice. (A) Body weight of NCoR1^{skm+/+} and ^{skm-/-} mice was measured before and after 12-week-treatment with HFD (n=8-10, each group). (B) Relative weight of tissues normalized by body weight in NCoR1^{skm+/+} and ^{skm-/-} mice (n=8, each group). (C) Representative macroscopic appearance of soleus and gastrocnemius muscles from NCoR1^{skm+/+} and ^{skm-/-} mice. (D) Intraperitoneal glucose tolerance test on mice on CD (left) and HFD (right) for 16 wks. The bar graphs represent the average area under the curve (AUC) (n=8). (E) Intraperitoneal insulin tolerance test in mice fed HFD for 18 wks (n=7-8). (F) Circadian activity, measured as the total ambulatory locomotor activity, and energy expenditure was evaluated by the measurement of oxygen consumption (VO₂), and by the calculation of the respiratory exchange ratio (RER) over a 24 hr period with CD-fed in NCoR1^{skm+/+} and ^{skm-/-} mice. The bar graphs represent the average for each group (n=12). Error bars represent SEM and P values were calculated with the Student t test and are indicated as *, P<0.05, **, P<0.01; ***, P<0.001. Data are expressed as mean±SEM.

FIG. 4: Histological Analyses of the Muscles of Control and NCoR1^{skm-/-} Mice.

(A) Histological analysis of gastrocnemius sections stained with H&E. (B) Distribution and mean diameter of muscle fibers in gastrocnemius and soleus. (C and D) Histological analysis of gastrocnemius and soleus sections by succinate dehydrogenase (C) and cytochrome C oxidase staining (D). (C). S, soleus; G, gastrocnemius. The ratio of the stained fibers is indicated in the graph.

FIG. 5: the Exercise Capacity is Enhanced in NCoR1^{skm-/-} Mice and there is no Difference in Cardiac Function Between NCoR1^{skm+/+} and ^{skm-/-} Mice.

(A, C) Schematic representation of protocols used for regular endurance exercise test (A) and the VO₂ₘₐₓ running experiment (C). (B) Running distance and cumulative number of shocks until exhaustion in individual animals fed a HFD (n=5-6). (D) VO₂ₘₐₓ and RER levels at the basal and at the VO₂ₘₐₓ condition in chow diet-fed mice (n=9, each group). Values are comparable to those obtained under HFD (see FIG. 7C). (E, F) Evaluation of heart rate (HR) (E) and blood pressure (BP) (F) in NCoR1^{skm+/+} and ^{skm-/-} mice (n=10). (G) Representative M-mode picture of echocardiography of NCoR1^{skm+/+} and ^{skm-/-} mice (n=8). Error bars represent SEM and P values were calculated with the Student t test and are indicated as *, P<0.05, **, P<0.01; ***, P<0.001.

FIG. 6: Histological Analyses of the NCoR1^{skm+/+} and ^{skm-/-} Gastrocnemius, and the Effects of gei-8 Knockdown in the Control Worms.

(A) Toluidine blue staining of gastrocnemius was performed in NCoR1^{skm+/+} and ^{skm-/-} mice. (B) The effects of RNAi inactivation of gei-8 in worms caning the ₚmyo-3MYO-3::GFP translational fusion highlighting myosin heavy chain.

FIG. 7: Histological Analyses of the Muscles of Mice and C. elegans.

(A) Transmission electron microscopy of non-oxidative fibers of NCoR1^{skm+/+} and ^{skm-/-} gastrocnemius. M, mitochondria. (B) Relative mitochondrial DNA content (Cox2 or 16S) in gastrocnemius was measured and normalized by genomic DNA content (Ucp2 and Hk) (n=6). (C) Representative MyHC1, 2a, and 2b immunohistochemical detection on serial sections of the soleus and gastrocnemius. (D) Subtypes of MyHCs in gastrocnemius and quadriceps were analyzed by qRT-PCR in NCoR1^{skm+/+} and ^{skm-/-} mice (n=6). (E) Indentity/similarity (%) in the sequences of gei-8 with mammalian NCoR1. Multiple alignment of SANT domains of NCoR1 homologs. Color identifies similarity. (F) Representative pictures of the effects of RNAi-mediated knockdown of gei-8 on mitochondrial morphology and number in a C. elegans strain carrying a mitochondrial GFP-reporter driven by the muscle-specific myo-3 promoter (left panel). Quantification of the mitochondrial induction by fluorescence upon gei-8 knockdown (middle panel), and of the efficacy of the RNAi-mediated gei-8 knockdown by qRT-PCR analysis (right panel). (G) Muscle-specific RNAi inhibition of gei-8 enhances respiration in C. elegans. Data are expressed as mean±SEM.

FIG. 8: Vascularization is Enhanced in Gastrocnemius Muscle of NCoR1^{skm-/-} mice. (A) mRNA levels of ERRs, PPARs, and angiogenesis-related genes are determined by qRT-PCR in NCoR1^{skm+/+} and ^{skm-/-} gastrocnemius (n=10). (B) Vegfa is increased in the gastrocnemius and soleus muscles of NCoR1^{skm-/-} mice. Assessment by qRT-PCR of mRNA levels of Vegfa isoforms, Vegfa-121, -165, and -189, in the quadriceps and soleus muscles of NCoR1^{skm+/+} and ^{skm-/-} mice (n=10-11). (C) Gastrocnemius muscles from NCoR1^{skm+/+} and ^{skm-/-} mice were stained with PECAM-1 antibody. Representative pictures are shown from three samples. Error bars represent SEM and P values were calculated with the Student t test and are indicated as *, P<0.05, **, P<0.01; ***, P<0.001.

FIG. 9: Identification of NcoR1-correlated genes. (A) Expression of NCoR1 mRNA in lung tissue of the different BXD strains (upper) and in muscle tissue from an F2 intercross between C57BL/6J and C3H/HeJ (lower panel). Natural expression variation across the animals is 1.5 fold in each tissue. (B) Pearson's r and Spearman's rank correlation coefficient, rho, were calculated with corresponding p values for the mRNA covariation between NCoR1 and genes involved in oxidative phosphorylation (Cycs, Cs, and Pdk4), mitochondrial uncoupling (Ucp3), fatty acid metabolism (Lcad), angiogenesis (Vegfb), glucose uptake (Glut4), and myogenesis (Mef2d, Mb, Mck). The tissue from which data were generated is indicated. (C) Gene expression analysis by qRT-PCR in NCoR1^{skm+/+} and ^{skm-/-} gastrocnemius (n=10). Data are expressed as mean±SEM.

FIG. 10: Increased PPARβ/δ and ERR Activity in NCoR1^{skm-/-} Muscle.

(A, B, E, F, and H) NCoR1 recruitment to the PPREs on mouse Ucp3 promoter and to the ERR-RE on human and mouse Pdk4 promoter determined by ChIP in NIH-3T3 cells transfected with an NCoR1-FLAG vector or in C2C12 myotubes. A schematic of the promoters of the Ucp3 (A) and Pdk4 (E) genes and the sequence alignment of the mouse, rat and human Pdk4 promoter is also shown to highlight the conservation of the NR1/2 (or ERR-RE) (E). Boxes indicate putative PPREs in the Ucp3 and the NR1/2, IRS, and Sp1 in the Pdk4 promoter. ChIP experiments for the Ucp3 promoter were performed in C2C12 myotubes both before and 6 hr after addition of a PPARIβ/δ (100 nM GW501516); not detected. ChIP experiments in HEK293 cells transfected with FLAG-NCoR1 and HA-ERRα vector (H). (C and G) Binding of acetylated histone 4 (H4) to the PPREs on the Ucp3 and to the NR1/2 on the Pdk4 promoters in ChIP assays, using either immortalized NCoR1^{L2/L2}MEFs, infected with an adenovirus either expressing GFP or Cre recombinase, or C2C12 myotubes infected with the Ad-shNCoR1 virus. Representative data is shown from 3 experiments. (D) Interaction between PPARβ/δ and NCoR1 determined by in vitro co-IP experiments from HEK293 cells, in which NCoR1-FLAG and/or V5-PPARβ/δ are expressed. IP was performed with control IgG (lanes 1, 3, 5, and 7) or anti-FLAG antibody (lanes 2, 4, 6, and 8) and the immunoblot was developed with an anti-V5 antibody. PPARβ/δ co-immunoprecipitated by the anti-FLAG antibody is indicated by an arrow. Input samples are shown in lanes 9-12. Data are expressed as mean±SEM.

FIG. 11: Enhanced MEF2 Activity in NCoR1^{skm-/-} Muscle.

(A) Gene expression of myogenesis-related genes was measured by qRT-PCR in NCoR1^{skm+/+} and ^{skm-/-} quadriceps (n=10). (B, C, and D) Acetylation levels of MEF2D were determined by Western blot after immunoprecipitation with an Ac-Lys Ab from gastrocnemius (B), from NCoR1^{L2/L2}-MEFs infected with Ad-GFP or Ad-Cre recombinase (C, right), and from C2C12 myotubes infected with Ad-shLacZ or Ad-shNCoR1 (D). MEF2D expression in total protein extracts was shown in the lower panels. The expression of NCoR1 and actin in NCoR1^{L2/L2}-MEFs was also shown (C, left). (E) MEF2 target mRNAs determined by qRT-PCR in C2C12 myotubes infected with either Ad-shLacZ or Ad-shNCoR1 (n=6). (F) NCoR1 recruitment to the MEF2 site of the mouse Mb promoter determined by ChIP in C2C12 myotubes. (G) Binding of either global acetylated histone 4 (H4) or H4 acetylated on K16 (H4K16) to the MEF2 site of the Mb gene was evaluated by ChIP from C2C12 myotubes infected as in (E). Data are expressed as mean±SEM.

FIG. 12: The Effects of NCoR1 Knockdown in the Muscles.

(A) Covariation analysis of the expression levels of Mef2a, Mef2c, MyoD, myf5, myf6, and Mstn (myostatin) with the expression of NCoR1. Pearson's r correlation of mRNA expression between NCoR1 and Mef2A, Mef2C, MyoD, myf5, myf6, Mstn was evaluated in the BXD mouse strains. (B-D) Gene expression profiling is analyzed in C2C12 myotubes with an NCoR1 knockdown. mRNA expression levels of nuclear receptors, transcription factors, and co-regulators (B), of proteins involved in mitochondrial function (C), and angiogenesis (D) were measured by qRT-PCR in C2C12 myotubes infected with Ad-sh LacZ (control) or Ad-sh NCoR1 (n=6). (E) Upregulation of the expression of Mef2d in the absence of NCoR1. Assessment of Mef2a, c, and d mRNA levels by qRT-PCR in quadriceps muscle of NCoR1^{skm+/+} and ^{skm-/-} mice (n=11). (F) Enhanced recruitment of acetylated histone 4 on the promoter region of the MEF2 target genes in the absence of NCoR1. Binding of histone H4 acetylated on K16 (H4K16) to the MEF2 binding sites of the Glut4 and Mck gene was evaluated by ChIP from C2C12 myotubes infected with either Ad-sh LacZ or Ad-sh NCoR1. A representative experiment of n=3 is shown. Error bars represent SEM and P values were calculated with the Student t test and are indicated as *, P<0.05, **, P<0.01; ***, P<0.001.

FIG. 13: Localization and Expression of NCoR1 in Physiological Condition.

(A-C) Localization of NCoR1 protein was determined either by immunofluorescence and quantified (A and B) and by western blot (C). 293T cells grown without (−) or with (+) 1 □M insulin for 1 hr were stained by DAPI or anti-NCoR1 (A). Quantification of nuclear NCoR1 is shown in (B). Nuclear and cytosolic fractions were separated from FLAG-NCoR1 transfected 293T cells after a 1 hr-stimulation without (−) or with 1 μM insulin (+) and protein levels were determined by western blotting (C). (D) mRNA levels of NCoRs and its target genes determined by qRT-PCR in MEFs cultured for 24 hr in 5 or 25 mM glucose (n=6). (E) NCoR1 protein was determined by western blotting from MEFs cultured for 24 hr in 5 or 25 mM glucose. (F) NCoR1 and SMRT mRNA was determined in MEFs cultured for 48 hr in 0, 5, and 25 mM glucose. (G) NCoR1 protein was determined by western blotting from MEFs cultured for 24 hrs in 0, 5, or 25 mM glucose. (H) NCoR1 mRNA expression in MEFs grown for 48 hr in 0 mM glucose with or without 0.03 mM oleic acid (n=4). (I) NCoR1 protein determined by western blotting from MEFs cultured as indicated in (G). (J) NCoR1 mRNA measured by qRT-PCR in muscles of resting mice or 3 hrs after an endurance run (14-wk old; n=5), in mice that were fed for 20 wks either with HFD or CD (28-wk old; n=8), in mice that were fasted or fed for 16 hrs (14-wk old; n=10), and in 6-month or 2-year old mice (n=10). (K) Model schematizing how different levels of NCoR1 controls transcription of muscle genes by controlling the activity of transcription factors (TFs; i.e. PPARβ/δ, ERR, and MEF2). Data are expressed as mean±SEM.

FIG. 14: Localization and Expression of NCoR1 in Physiological Condition.

(A) Localization of NCoR1 is determined by the immunofluorescence experiments. 293T cells, transfected with FLAG-NCoR1 vector were stained by DAPI, anti-NCoR1 or anti-FLAG antibody, in cells after 1-hr with or without 1 μM insulin. (B) mRNA levels of NCoRs and their target genes were determined by qRT-PCR in C2C12 myotubes cultured for 24 hrs in 25 mM or 0 mM glucose (n=6). (C and D) NCoR1 mRNA (C) and protein (D) levels were determined in epidydimal white adipose tissue in mice fed for 20 weeks either with HFD or CD (28-wk old; n=8, 9). Error bars represent SEM and P values were calculated with the Student t test and are indicated as *, P<0.05, **, P<0.01; ***, P<0.001.

### DETAILED DESCRIPTION OF THE INVENTION

The invention is based in part upon the discovery that NCoR1^{-/-} (NCoR1^{skm-/-}) mice display a remarkable enhanced exercise capacity. This enhanced exercise capacity was the result of increased muscle mass and a muscle fiber type shift towards more oxidative fibers, coordinated by the induction of genes involved in mitochondrial biogenesis and function, ensuing from the activation of PPARIβ/δ, ERR, and MEF2.

Transcriptional coregulators control the activity of many transcription factors and are thought to have wide ranging effects on gene expression patterns. As described herein, muscle-specific nuclear receptor corepressor 1 (NCoR1) knockout mice have rather selective phenotypic changes, characterized by enhanced exercise endurance due to an increase of both muscle mass and of mitochondrial number and activity. The activation of selected transcription factors that control muscle function, such as the myocyte enhancer factor 2, the peroxisome proliferator-activated receptor β/δ and the estrogen-related receptors, underpinned these phenotypic alterations. NCoR1 levels are decreased in conditions that require fat oxidation resetting transcriptional programs to boost oxidative metabolism. The capacity of NCoR1 to modulate oxidative metabolism may be conserved as the knockdown of gei-8, the sole C. elegans NCoR homolog, also robustly increased muscle mitochondria and respiration. Collectively, our data demonstrate that NCoR1 plays an adaptive role in muscle physiology and that interference with NCoR1 action could be used to improve muscle function.

Accordingly the invention features methods of increasing muscle mass or muscle mitrochondrial oxidative metabolism by administering to a subject a compound that decreases muscle-specific nuclear receptor corepressor 1 (NCoR1) expression of activity. Also included in the invention are methods of increasing of mitochondrial number and function in a cell by contacting the cell a compound that decreases muscle-specific nuclear receptor corepressor 1 (NCoR1) expression of activity.

The invention further provides methods of treating, alleviating a symptom or delaying of a disorder associated with mitochondrial dysfunction by administering to a subject a compound that decreases muscle-specific nuclear receptor corepressor 1 (NCoR1) expression of activity. Disorders associated with mitochondrial dysfunction include genetic mitochondrial disease or metabolic disorders.

The invention further provides methods of treating, alleviating a symptom or delaying of a muscular dystrophy disorder by administering to a subject a compound that decreases muscle-specific nuclear receptor corepressor 1 (NCoR1) expression of activity.

The subject is suffering from or susceptible to developing the disorder. The compound is administered or the cell is contacted in an amount sufficient to activate the myocyte enhancer factor 2, the peroxisome proliferator-activated receptor β/δ, or an estrogen related receptor.

Compounds that decrease NCoR1 expression or activity are referred to herein as a NCoR1inhibitor. The NCoR1inhibitor can be administered alone or in combination.

A decrease in NCoR1 expression or activity is defined by a reduction of a biological function of the NCoR1 protein. A NCoR1 biological function includes for example, the repression of transcription of nuclear hormone receptors. NCoR1 expression is measured by detecting a NCoR1 transcript or protein. NCoR1inhibitor are known in the art or are identified using methods described herein.

The NCoR1inhibitor inhibitor is for example an antisense NCoR1 nucleic acid, a NCoR1 nspecific short-interfering RNA, or a NCoR1 nspecific ribozyme.

By the term “siRNA” is meant a double stranded RNA molecule which prevents translation of a target mRNA. Standard techniques of introducing siRNA into a cell are used, including those in which DNA is a template from which an siRNA RNA is transcribed. The siRNA includes a sense NCoR1 nucleic acid sequence, an anti-sense NCoR1 nucleic acid sequence or both. Optionally, the siRNA is constructed such that a single transcript has both the sense and complementary antisense sequences from the target gene, e.g., a hairpin.

Binding of the siRNA to a NCoR1 transcript in the target cell results in a reduction in NCoR1 production by the cell. The length of the oligonucleotide is at least 10 nucleotides and may be as long as the naturally-occurring NCoR1 transcript. Preferably, the oligonucleotide is 19-25 nucleotides in length. Most preferably, the oligonucleotide is less than 75, 50, 25 nucleotides in length.

Other examples of molecules decrease NCoR1 expression or activity includes and NCoR1 antibodies or compound that inhibits or dissociates the NCoR1-histone deacetylase (HDAC) complex. For example the compound is an HDAC inhibitor. HDAC inhibitors are well known in the art.

The present invention provides for both prophylactic and therapeutic methods of treating a subject at risk of (or susceptible to) a disorder or having a disorder associated with aberrant (e.g., insufficient) mitochondrial function. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

Muscle mass is increased or oxidative metabolism is promoted by exposing, e.g., contacting a tissue or cell with a compound that decrease the expression or activity of NCoR1. By increasing muscle mass is meant that the subject has more muscle mass compared to a subject that has not been administered the compound. Muscle mass is measure by methods known in the art. In some aspects, there is a shift in muscle fiber type to more oxidative fibers. By promoting oxidative metabolism is meant an increase in oxygen consumption compared to a tissue or cell that has not been in contact with compound. Tissues or cells are directly contacted with compound. Alternatively, the compound is administered systemically. The compound is administered in an amount sufficient to increase (e.g., activate) myocyte enhancer factor 2, the peroxisome proliferator-activated receptor β/δ or estrogen-related receptors. Oxidative metabolism is measured by techniques known in the art, such as by the methods described herein.

The methods are useful to treat, alleviate the symptoms of, or delay the onset of a disorder associated with aberrant mitochondrial function. Disorders associated with aberrant mitochondrial function include for example metabolic disorders, neurodegenerative disorders aging related disorders and chronic inflammatory disorders. Mitochondrial disorders include also diseases with inherited and/or acquired mitochondrial dysfunction, such as Charcot-Marie-Tooth disease, Type 2A2, Mitochondrial Encephalopathy Lactic Acidosis and Stroke (MELAS), Leigh syndrome, Barth syndrome, Leber's optic neuropathy, Fatty acid oxidation disorders, Inherited forms of deafness and blindness, metabolic abnormalities induced by exposure to toxic chemicals and/or drugs (e.g. cisplatin induced deafness).

Metabolic disorders include for example, type II diabetes, obesity, hyperglycemia, glucose intolerance, insulin resistance (i.e., hyperinsulinemia, metabolic syndrome, syndrome X), hypercholesterolemia, hypertension, hyperlipoproteinemia, hyperlipidemia (e.g., dyslipidemia), hypertriglylceridemia, non-alcoholic fatty liver disease (NAFLD, e.g. hepatostatosis and steatohepatitis), cardiovascular disease, atherosclerosis, peripheral vascular disease, kidney disease, ketoacidosis, thrombotic disorders, nephropathy, diabetic neuropathy, diabetic retinopathy, sexual dysfunction, dermatopathy, dyspepsia, hypoglycemia, cancer or edema.

The methods are useful to treat, alleviate the symptoms of, or delay the onset of a muscular dystrophy. Muscular dystrophy disorders include both inherited forms of muscle dysfunction such as Duchenne's muscular dystrophy, Becker's dystrophy, Emery-Dreyfuss dystrophy, facioscapulohumeral muscular dystrophy, limb girdle syndromes, myotonic dystrophy and acquired forms of muscle weakness and sarcopenia (e.g. after disuse or drug induced).

Neurodegenerative disorders include diseases such as Dementia, Alzheimer's disease, Parkinson's disease, and Huntington's disease.

Chronic inflammatory diseases include disease such as celiac disease, vasculitis, lupus, chronic obstructive pulmonary disease (COPD), irritable bowel disease, atherosclerosis, arthritis, and psoriasis.

Aging related disorders includes disease such as cancer, dementia, ardiovascular disease, such as arteriosclerosis, hypertension, diabetes mellitus (type I or type II) arthritis, sarcopenia, muscle frailty, cataracts, Alzheimer's disease and osteoporosis.

The subject is suffering from or a susceptible to developing a metabolic disorder. Subjects suffering from or at risk of developing a metabolic disorder are identified by methods known in the art. For example diabetes is diagnosed by for example by measuring fasting blood glucose levels or insulin or by glucose tolerance test. Normal adult glucose levels are 60-126 mg/dl. Normal insulin levels are 7 mU/mL±3mU. Hypertension is diagnosed by a blood pressure consistently at or above 140/90. Cardiovascular disease is diagnosed by measuring cholesterol levels. For example, LDL cholesterol above 137 or total cholesterol above 200 is indicative of cardiovascular disease. Hyperglycemia is diagnosed by a blood glucose level higher than 10 mmol/l (180 mg/dl). Glucose intolerance is diagnosed by a two-hour glucose levels of 140 to 199 mg per dL (7.8 to 11.0 mmol) on the 75-g oral glucose tolerance test. Insulin resistance is diagnosed by a fasting serum insulin level of greater than approximately 60 pmol/L. Hypoglycemia is diagnosed by a blood glucose level lower than 2.8 to 3.0 mmol/L (50 to 54 mg/dl). NAFLD is diagnosed by liver fat accumulation detected by CT or MRI scanning or echography and the presence of abnormal liver function (SGOT and SGPT) tests. Obesity is diagnosed for example, by body mass index. Body mass index (BMI) is measured (kg/m² (or lb/in²×704.5)). Alternatively, waist circumference (estimates fat distribution), waist-to-hip ratio (estimates fat distribution), skinfold thickness (if measured at several sites, estimates fat distribution), or bioimpedance (based on principle that lean mass conducts current better than fat mass (i.e., fat mass impedes current), estimates % fat) is measured. The parameters for normal, overweight, or obese individuals is as follows: Underweight: BMI<18.5; Normal: BMI 18.5 to 24.9; Overweight: BMI=25 to 29.9. Overweight individuals are characterized as having a waist circumference of >94 cm for men or >80 cm for women and waist to hip ratios of ≧0.95 in men and ≧0.80 in women. Obese individuals are characterized as having a BMI of 30 to 34.9, being greater than 20% above “normal” weight for height, having a body fat percentage >30% for women and 25% for men, and having a waist circumference >102 cm (40 inches) for men or 88 cm (35 inches) for women. Individuals with severe or morbid obesity are characterized as having a BMI of 35.

The methods described herein lead to a reduction in the severity or the alleviation of one or more symptoms of the metabolic disorder. Symptoms of diabetes include for example elevated fasting blood glucose levels, blood pressure at or above 140/90 mm/Hg; abnormal blood fat levels, such as high-density lipoproteins (HDL) less than or equal to 35 mg/dL, or triglycerides greater than or equal to 250 mg/dL (mg/dL=milligrams of glucose per deciliter of blood). Efficacy of treatment is determined in association with any known method for diagnosing the metabolic disorder. Alleviation of one or more symptoms of the metabolic disorder indicates that the compound confers a clinical benefit.

The compounds, e.g., NCoR1inhibitors (also referred to herein as “active compounds”) of the invention, and derivatives, fragments, analogs and homologs thereof, can be incorporated into pharmaceutical compositions suitable for administration. Such compositions typically comprise the peptide or mimetic, and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Mitochondrial disorders are diagnosed for example in combination with abnormalities of glucose and lipid homeostasis, ketone bodies and abnormalities in acid/base balance and abnormal levels of other metabolites in the blood.

Neurodegenerative disorders are diagnosed for example by physical and neurological examination, family history, Electroencephalograms (EEGs) MRI and CAT scans.

Muscular dystrophy disorders are diagnosed for example, by a combination of “genetics diagnostics (family history, pre- and perinatal DNA analysis), abnormal enzyme levels (e.g. creatine phosphokinase (CPK) levels, lacatate dehydrogenase (LDH), SGOT), ECG and EMG, and analysis of muscle biopsies”.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NCoR1 inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated fully herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

The pharmaceutical compositions can be included in a container, pack, or dispenser together with instructions for administration.

### EXAMPLES

### Example 1

### General Methods

Animal Studies. Mouse Experiments.

NCoR1 floxed (NCoR1^{L2/L2}), NCoR1^{skm+/+} and ^{skm-/-} mice were generated at the Mouse Clinical Institute (Strasbourg, France) and phenotyped (Champy et al., 2004; Champy et al., 2008).

Generation of NCoR1 Floxed (NCoR1^{L2/L2}) Mice.

For the generation of NCoR1 floxed (NCoR1 ^{L2/L2}) mice, genomic DNA covering the NCoR1 locus was amplified from the 129Sv strain by using high-fidelity PCR. The resulting DNA fragments were assembled into the targeting vector (Institut Clinique de la Souris). The construct was then electroporated into 129Sv embryonic stem (ES) cells. G418-resistant colonies were selected and analyzed for homologous recombination by PCR and positive clones were verified by Southern blot hybridization. Thereafter, genomic DNA was prepared from ES cells, digested with EcoRI or SpeI, subjected to electrophoresis on a 0.8% agarose gel, and transferred to a positively charged nylon transfer membrane (Amersham Biosciences). The karyotype was verified and several correctly targeted ES cell clones were injected into blastocysts from C57BL/6J mice. These blastocysts were transferred into pseudopregnant females, resulting in chimeric offspring that were mated to female C57BL/6J mice that express the Flp recombinase under the control of the ubiquitous cytomegalovirus promoter (Rodriguez et al., 2000). Offspring that transmitted the mutated allele, in which the selection marker was excised, and that lost the Flp transgene (NCoR1^{L2/WT} mice) were selected, mated with human skeletal actin (HSA)-Cre mice, and then further intercrossed to generate premutant HSAcre^{Tg/0}/NCoR1^{L2/L2} mice. A PCR genotyping strategy was subsequently used to identify HSAcre^{Tg/0}/NCoR1^{L2/L2} and HSAcre^{0/0}/NCoR1^{L2/L2} mice.

Animal Procedures and Biochemical Measurements.

All mice were maintained in a temperature-controlled (23° C.) facility with a 12 hr light/dark cycle and were given free access to food and water. Regular chow diet and high-fat diet were obtained from UAR (Villemoison sur Orge, France), Research Diet (New Brunswick, N.J.), respectively. The control diet (EQ12310) contained 16.8% protein, 73.5% carbohydrate and 4.8% fat, whereas the high-fat diet (D12492) contained 26.2% protein, 26.3% carbohydrate and 34.9% fat. The mice were fasted 4h before harvesting blood for subsequent blood measurements, and tissues for RNA isolation, lipid measurements and histology (Champy et al., 2004; Champy et al., 2008). Indirect calorimetry to monitor O₂ consumption, CO₂ production, and measurement of activity was measured using Comprehensive Lab Animal Monitoring System (CLAMS) (Columbus Instruments, Columbus, Ohio) (Watanabe et al., 2006).

OGTT and ipGTT was performed in animals that were fasted overnight. Glucose was administered by gavage or intraperitoneal injection at a dose of 2 g/kg BW. ipITT was done in 4h fasted animals. Insulin was injected at a dose of 0.50 U/kg BW. Glucose quantification was done with the Maxi Kit Glucometer 4 (Bayer Diagnostic, Puteaux, France) or Glucose RTU (bioMérieux Inc., Marcy l'Etoile, France). Plasma insulin concentrations were measured using ELISA for mouse (Cristal Chem Inc., Downers Grove, Ill.) or IRI for human samples. Free fatty acids, triglycerides, total cholesterol, LDL and HDL cholesterol were determined by enzymatic assays (Roche, Mannheim, Germany).

The systolic and diastolic blood pressure and heart rate was measured by a computerized tail-cuff system (BP-2000, Visitech Systems, Apex, N.C.) in conscious animals (Koutnikova et al., 2009). Following 10 preliminary measurements in pre-warmed tail cuff (36° C.) device to accustom mice to the procedure, 10 actual measurements cycles were collected on 5 consecutive days at fixed diurnal interval and averaged for each individual animal. As movement artifact could reduce the number of successful when 7 out of 10 measurements were valid with a standard deviation less than 10 mmHg. HR was also monitored in the procedure. For each individual, the average value of BP and HR in the last 2 days was used for analysis. Echocardiography was performed in 14-week-old male mice using a using a Vevo2100 system (Visualsonics, Toronto, Canada) and a 40 MHz linear transducer. Mice were anesthetized with isoflurane (2% in O₂), placed on a heating table and the chest area was shaved. The ultrasound probe was fixed on a supporting stand and set manually in a parasternal short-axis view position. Left ventricular anterior and posterior wall motion and thickness, as well as ventricular diameters were evaluated in at least three images acquired in conventional 2D-guided M-mode (200 mm/s). Fractional shortening (FS) and ejection fraction (EF) were calculated according to the Teichholz formula (Gardin et al., 1995; Jakobsen et al., 2006).

Endurance exercise experiments to analyze the expression of NCoR1 in wild type C57B16J mice were performed exactly, as described (Canto et al., 2009).

C. elegans Experiments.

C. elegans strains were cultured at 20° C. on nematode growth media agar plates seeded with E. coli strain OP50 unless stated otherwise. Strains used were 5.14103 (zcIs14[myo-3::GFP(mit)]), NR350 kzIs2O[pDM#715(hlh-1p::rde-1)+pTG95(sur-5p::nls::GFP)] and RW1596 stEx3O[myo-3p::GFP+rol-6(su1006)]. Strains were provided by the Caenorhabditis Genetics Center (University of Minnesota). The SJ4103 strain was used to highlight mitochondria in body wall muscle (Benedetti et al., 2006). The NR350 strain was used to specifically knockdowned by RNAi gei-8 in body wall muscle (Durieux et al., 2011). The RW1596 strain was used as a control for pmyo-3::gfp expression in response to gei-8 inhibition (Herndon et al., 2002).

The protein most homologous to mouse NCoR1 was identified by using a protein blast search of the WormBase site (http://www.wormbase.org/db/searches/blast_blat) and NCBI Blast of the NCBI site (http://www.ncbi.nlm.nih.gov/blast/Blast.cgi). Multiple sequence alignment was performed with the Clustal W software (http://www.ebi.ac.uk/Tools/msa/clustalw2/).

Bacterial feeding RNAi experiments were carried out essentially as described previously (Kamath et al., 2001). The clones used were gei-8 (C14B9.6) and HT115, carrying the empty vector RNAi L4440, as a control. The reduction of the gei-8 mRNA expression quantified by qRT-PCR was 30% at the L4 stage. Clones were purchased from GeneService Ltd and those were confirmed.

GFP expression and quantification was carried out according to the protocol previously described (Durieux et al., 2011). Briefly, GFP was monitored in Day 3 adults. Fluorimetric assays were performed using a Victor X4 multilabel plate reader (Perkin-Elmer Life Science). Eighty roller worms were picked at random (20 worms per well of a black-walled 96-well plate) and each well was read four times and averaged. Each experiment was repeated at least twice.

For picture acquisition, animals were mounted on 2% agarose pads in a droplet of 10 mM tetramisole (Sigma) and examined using a Zeiss Axioplan-2 microscope (Carl Zeiss MicroImaging, Thornwood, N.Y., USA) equipped for both DIC and epifluorescence. Images were obtained using a Coolsnap ES² camera. 20 worms were observed for each condition and two rounds of observation were performed independently.

Oxygen consumption was measured using the Seahorse XF24 equipment (Seahorse Bioscience Inc., North Billerica, Mass.). Typically, 200 two-day old animals per conditions were recovered from NGM plates with M9 medium, washed three times in 2 mL M9 to eliminate residual bacteria, and resuspended in 500 μL M9 medium. Worms were transferred in 24-well standard Seahorse plates (#100777-004) (50 worms per well) and oxygen consumption was measured 6 times. Respiration rates were normalized to the number of worms in each individual well and repeated at least twice.

Histological and EM Analyses.

Staining of muscles with hematoxylin/eosin, immunohistochemical and EM analysis, analysis of enzymatic activity of SDH and COX was carried out as described (Lagouge et al., 2006). Specifically, for immunohistochemical analysis, cryo-sections of the indicated snap frozen muscles were stained with anti-PECAM-1 antibody (1:100, eBioscience), and anti-MyHC1, MyHC2a and MyHC2b antibodies, purified from the cell culture supernatant of BA-D5, SC-71 and BF-F3 hybridomas, respectively (Deutsche Sammlung von Mikroorganismen and Zellkulturen GmbH). Adjacent sections were used for staining with MyHCs. Enzymatic staining for COX activity was performed as described (Seligman et al., 1968).

mRNA Analysis and Identification of NcoR1-Correlated Genes.

The mRNA expression levels were measured in cells and tissues using qRT-PCR (Lagouge et al., 2006). List of primer sets used for qRT-PCR are provided in Table 1.

TABLE 1
List of primer sets used for qRT-PCR.

| Gene | | SEQ | | SEQ |
|---|---|---|---|---|
| name | Forward Primer | ID NO | Reverse Primer | ID NO |
| 36B4 | AGATTCGGGATATGCTGTTGG | 1 | AAAGCCTGGAAGAAGGAGGTC | 2 |
| Angiopoietin-2 | AGAAGAGCAAACCACCTTCAGAG | 3 | GTCACAGTAGGCCTTGATCTCC | 4 |
| CD36 | GATGTGGAACCCATAACTGGATTCAC | 5 | GGTCCCAGTCTCATTTAGCCACAGTA | 6 |
| Citrate | GGAGCCAAGAACTCATCCTG | 7 | TCTGGCCTGCTCCTTAGGTA | 8 |
| synthase | | | | |
| c-jun | CCTGTCCCCTATCGACATGG | 9 | CTTTTCCGGCACTTGGAGG | 10 |
| CoxIV | TGGGAGTGTTGTGAAGAGTGA | 11 | GCAGTGAAGCCGATGAAGAAC | 12 |
| CPT1b | CCCATGTGCTCCTACCAGAT | 13 | CCTTGAAGAAGCGACCTTTG | 14 |
| Cytochrome C | TCCATCAGGGTATCCTCTCC | 15 | GGAGGCAAGCATAAGACTGG | 16 |
| ERR□ | ACTGCCACTGCAGGATGAG | 17 | CACAGCCTCAGCATCTTCAA | 18 |
| ERR□ | GGGAGCTTGTGTTCCTCATC | 19 | ATCTCCATCCAGGCACTCTG | 20 |
| ERR□ | GATGAGCCTCCTCCAGAGTG | 21 | TGCACAGCTTCCACATCTTC | 22 |
| FGF | TCCTCATTCCAAGAAACTCTGTCC | 23 | GGTAAGAGTGGTCTTCTGTCCC | 24 |
| FGFR-2 | GGAGGCTATAAGGTACGAAACCAG | 25 | GACCCGTATTCATTCTCCACCA | 26 |
| FLT-1 | GCTCTGATGACCGAACTCAA | 27 | ATTCCACGATCACCATCAGA | 28 |
| GLUT1 | ACTGGGCAAGTCCTTTGAGA | 29 | GTCTAAGCCAAACACCTGGGC | 30 |
| GLUT4 | CTTCTTTGAGATTGGCCCTGG | 31 | AGGTGAAGATGAAGAAGCCAAGC | 32 |
| HIF-1□ | TCCATGTGACCATGAGGAAA | 33 | CTTCCACGTTGCTGACTTGA | 34 |
| LCAD | GTAGCTTATGAATGTGTGCAACTC | 35 | GTCTTGCGATCAGCTCTTTCATTA | 36 |
| MCK | TGAACTCGCCCGTCAGGCTGTTGAG | 37 | GATGTCATCCAGACTGGGGTGGACAACC | 38 |
| MEF2A | GTAGCGGAGACTCGGAATTG | 39 | ACCTTCTAGTCCAGGGCTGC | 40 |
| MEF2B | GATGCAGCTGAAGGGAAAGA | 41 | GTCACCTGCCTGTTCCTTTG | 42 |
| MEF2C | TCCATCAGCCATTTCAACAA | 43 | GTTACAGAGCCGAGGTGGAG | 44 |
| MEF2D | CTTCGCGTAACCGAGGATT | 45 | GGGACTAGTGATCAGTTCATGG | 46 |
| MyHC 1 | CCAAGGGCCTGAATGAGGAG | 47 | GCAAAGGCTCCAGGTCTGAG | 48 |
| MyHC 2b | ACAAGCTGCGGGTGAAGAGC | 49 | CAGGACAGTGACAAAGAACG | 50 |
| MyHC 2a | AAGCGAAGAGTAAGGCTGTC | 51 | GTGATTGCTTGCAAAGGAAC | 52 |
| MyHC 2x | CCAAGTGCAGGAAAGTGACC | 53 | AGGAAGAGACTGACGAGCTC | 54 |
| Myoglobin | CTGACGAAGGCCACTTTGCACCTCTG | 55 | GCACAAGATCCCGGTCAAGTACCTGGAG | 56 |
| NCoR1 set 1 | CTGGTCTTTCAGCCACCATT | 57 | CCTTCATTGGATCCTCCATC | 58 |
| NCoR1 set 2 | CGCTGCAGGAGAGGTTTATC | 59 | CCTGCATCTGCTGTGAGGTA | 60 |
| Nur77 | GGTGTTGATGTTCCCGCCT | 61 | TCAGTGATGAGGACCAGAGCG | 62 |
| PDGF-B | CCACTCCATCCGCTCCTTT | 63 | AAGTCCAGCTCAGCCCCAT | 64 |
| PDK4 | AAAGGACAGGATGGAAGGAATCA | 65 | ATTAACTGGCAGAGTGGCAGGTAA | 66 |
| PGC-1□ | AAGTGTGGAACTCTCTGGAACTG | 67 | GGGTTATCTTGGTTGGCTTTATG | 68 |
| PGC-1□ | TGGAGACTGCTCTGGAAGGT | 69 | TGCTGCTGTCCTCAAATACG | 70 |
| PPAR□ | CCTGAACATCGAGTGTCGAATAT | 71 | GGTTCTTCTTCTGAATCTTGCAGCT | 72 |
| PPAR\tilde{□}□ | CTCTTCATCGCGGCCATCATTCT | 73 | TCTGCCATCTTCTGCAGCAGCTT | 74 |
| PPAR□1 | ATGGGTGAAACTCTGGGAGATTCT | 75 | CTTGGAGCTTCAGGTCATATTTGTA | 76 |
| SDH | GGACCTATGGTGTTGGATGC | 77 | GTGTGCACGCCAGAGTATTG | 78 |
| SMRT | TGTCACCTCAGCCAGCATAG | 79 | CGCCGTAAGTAGTCCTCCTG | 80 |
| UCP2 | TGGCAGGTAGCACCACAGG | 81 | CATCTGGTCTTGCAGCAACTCT | 82 |
| UCP3 | ACTCCAGCGTCGCCATCAGGATTCT | 83 | TAAACAGGTGAGACTCCAGCAACTT | 84 |
| VEGF-A 121 | AACGATGAAGCCCTGGAGTG | 85 | TGAGAGGTCTGGTTCCCGA | 86 |
| VEGF-A 165 | AACGATGAAGCCCTGGAGTG | 87 | GACAAACAAATGCTTTCTCCG | 88 |
| VEGF-A 189 | AACGATGAAGCCCTGGAGTG | 89 | AACAAGGCTCACAGTGAACG | 90 |
| VEGF-B | AGCCACCAGAAGAAAGTGGT | 91 | GCTGGGCACTAGTTGTTTGA | 92 |

The GeneNetwork program (http://www.genenetwork.org) was used to generate a broad range of candidate genes that correlate with NCoR1 and may contribute to the phenotype of NCoR1^{skm-/-} mice. Skeletal muscle mRNA expression from an Agilent microarray platform was analyzed across 124 females from a classic F2 intercross between C57BL/6J and C3H/HeJ [UCLA BHHBF2 Muscle; van Nas et al. (2010); GEO GSE12795]. The sole NCoR1 marker on the Agilent muscle database was selected (10024414685, 3′ UTR) and compared across all other transcripts to find covariates. Lung mRNA expression from Affymetrix M430 2.0 microarrays in a recombinant inbred intercross between C57BL/6J and DBA/2J was analyzed across 51 strains (HZI BXD Lung M430v2 (April 2008) RMA). Four NCoR1 probe sets from this microarray were selected: 1423200_at (3′ UTR), 1435914_at (3′ UTR), 1423202_a_at (exonic and 3′UTR), and 1423201_at (exonic). For all four probe sets, the top 2000 Pearson's trait correlations within the same microarray were calculated. Strong correlates were selected for validation by qRT-PCR (e.g. Mck; r=−0.63 and Mef2d; r=0.73, both p<0.001). Further correlations were directly calculated for genes with key metabolic roles, and genes with significant correlations were selected for qRT-PCR as well (e.g. Cs; r=0.35, p=0.01).

Cell Culture and Adenoviral Infections.

Mouse embryonic fibroblasts (MEFs) from NCoR1^{L2/L2} floxed mice were prepared and immortalized. Then, GFP- or Cre recombinase-expressing adenovirus (Ad-GFP or -Cre) were infected at a MOI=20 to generate NCoR1^{+/+} or ^{-/-} MEFs, respectively. C2C12 skeletal muscle cells were grown and differentiated as described previously (Lagouge et al., 2006). In order to knockdown NCoR1 expression, two different shRNA containing adenoviruses were made by BLOCK-iT Adenoviral RNAi Expression System (Invitrogen) and each set of oligonucleotides was used following the manufacturer's instructions:

Set 1:
FWD:
(SEQ ID NO.: 93)
5′-CACCGCGTCAGCTTTCTGTGATTCCCCAAGGAATCACA
GAAAGCTGACGC-3′
REV:
(SEQ ID NO.: 94)
5′-AAAAGCGTCAGCTTTCTGTGATTCCTTCGGGAATCACAG
AAAGCTGACGC-3′
Set 2:
FWD:
(SEQ ID NO.: 95)
5′-CACCGGGTCTATCTCTCTGGGATTGCGAACAATCCCAGAG
AGATAGACCC-3′
REV:
(SEQ ID NO.: 96)
5′-AAAAGGGTCTATCTCTCTGGGATTGTTCGCAATCCCAGAG
AGATAGACCC-3′

C2C12 cells were infected with either these two different shRNA-containing adenoviruses or shRNA for LacZ (control) at a MOI=20.

ChIP, Co-IP Experiments, and Western Blot Analyses.

In one set of ChIP experiments to evaluate the recruitment of NCoR1 to the promoters of the mouse Pdk4 and Ucp3 genes, we used NIH-3T3 cells (10×10⁶ cells) transfected with pCMX-NCoR1-FLAG, pcDNA3-VTS-mPPAR□ (V5-tagged PPARβ/δ expression vector), and pcDNA-HA-ERRα with lipofectamine 2000 according to the manufacturers protocol. We also used pCMX-NCoR1-FLAG- and pcDNA-HA-ERRα-transfected HEK293 cells for the analysis of human Pdk4 promoter. We adapted the ChIP protocol described previously (Metivier et al., 2008). In another set of experiments, we used non-transfected C2C12 myotubes and a homemade NCoR1 polyclonal antibody to detect the recruitment of endogenous NCoR1 to known PPARβ/δ and ERRγ binding sites of Ucp3, and Pdk4. For the ChIP experiments to analyze the binding of acetylated histone 4 (H4), we used the immortalized NCoR1^{L2/L2} mouse embryonic fibroblasts (MEFs; 10×10⁶ cells), infected with adenovirus (Ad-GFP or Ad-Cre recombinase) to generate the control and NCoR1 knockdown-MEFs. Two days after the infection, these cells were used for ChIP experiments and determination of MEF2D protein expression and acetylation. For ChIP experiments to analyze the binding of either global acetylated histone 4 (H4) or histone H4 acetylated on K16 (H4K16) to the MEF2 binding sites of the mouse Mb, Mck, and Glut4 genes we used C2C12 myotubes (4×10⁶ cells) infected with either an Ad-sh LacZ (as a control) or Ad-sh NCoR1 (to knock-down NCoR1) adenovirus.

For Co-IP studies, NCoR1-FLAG1 and V5-PPARβ/δ or HA-ERRα overexpressing HEK293 cells were used. Immunoprecipitation was performed with 3 μg of antibody [anti-FLAG Ab (Sigma-Aldrich), anti-V5 Ab (Invitrogen), and anti-HA Ab (Abcam)] and the resulting immunoprecipitates was used for Western blot analysis.

For the acetylation assays of MEF2D, we used the immortalized NCoR1^{L2/L2} MEFs infected with Ad-GFP or Ad-Cre recombinase as mentioned above. Two days after infection, total cell lysate was obtained and immunoprecipitated with 5 μg of anti-acetyl lysine antibody (Cell Signaling). These samples were used for western blot analysis with anti-MEF2D Ab (Becton, Dickinson and Company).

Statistical Analyses.

Statistical analyses were performed with a Student's t test for independent samples. Data are expressed as mean±SEM, and p values smaller than 0.05 were considered as statistically significant. Statistical significance is displayed as * (p<0.05), ** (p<0.01), or *** (p<0.001).

### Example 2

### NCoR1^{SKM-/-} Mice have Increased Muscle Mass

Given the embryonic lethality of germline NCoR1^{-/-} mice [(Jepsen et al., 2000), Table 2], we generated a floxed NCoR1 mouse line in which exon 11 of the NCoR1 gene (Horlein et al., 1995) was flanked with LoxP sites, priming it for subsequent deletion using the Cre-LoxP system.

TABLE 2
Number of the germline NCoR1 wildtype, heterozygous,
and knockout mice that are born from heterozygous matings.

| | Number of | |
|---|---|---|
| Genotype | pups | (%) |
| NCoR1^{WT/WT} | 31 | 39.7 |
| NCoR1^{L-/WT} | 47 | 60.3 |
| NCoR1^{L-/L-} | 0 | 0.0 |

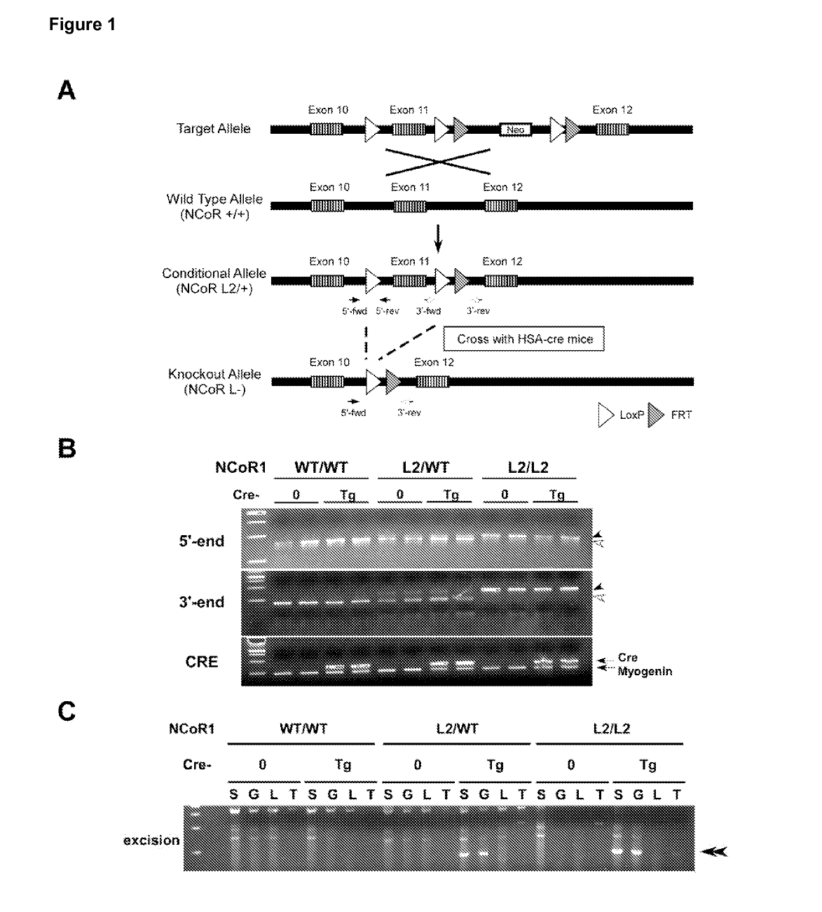

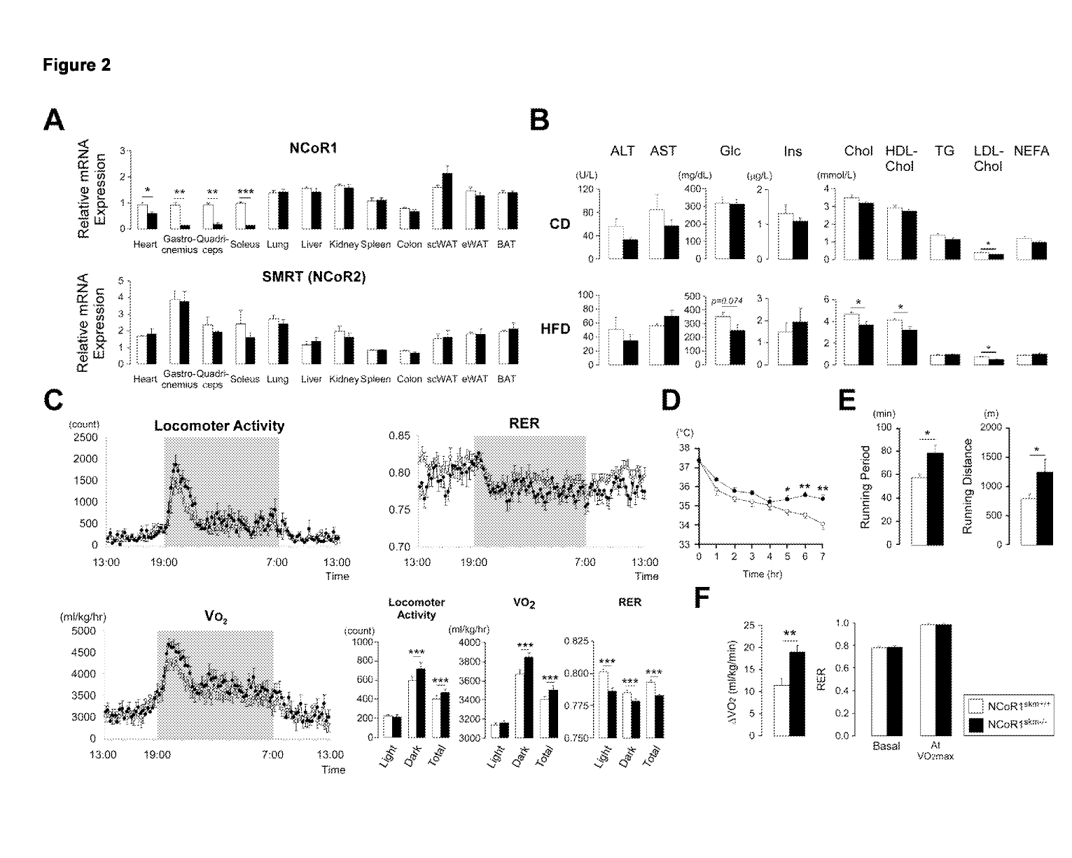

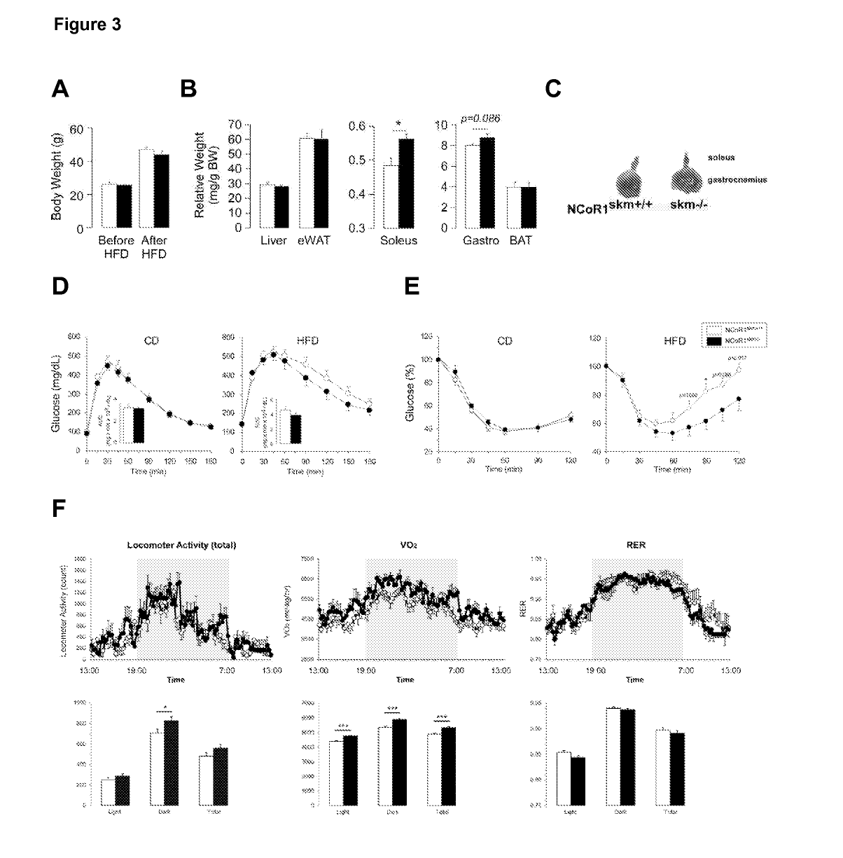

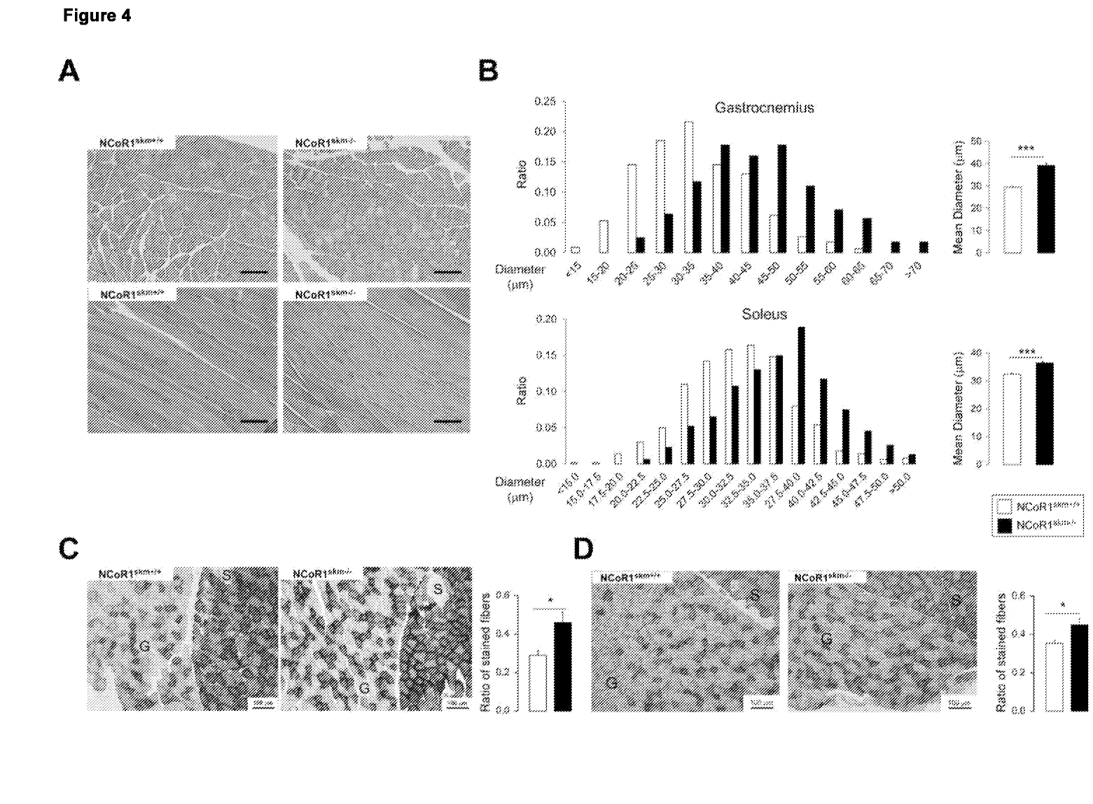

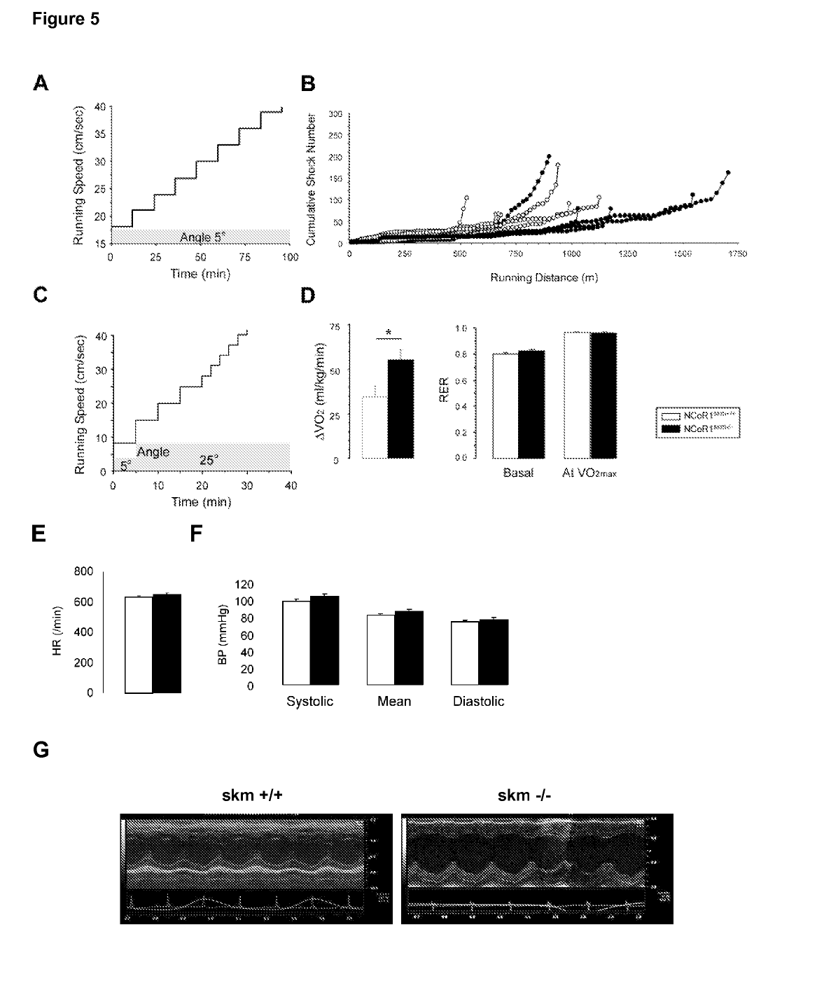

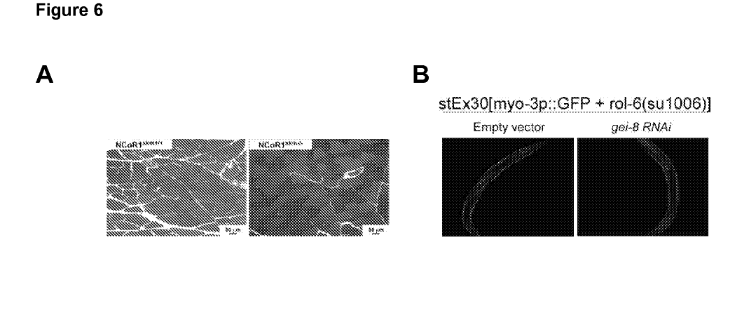

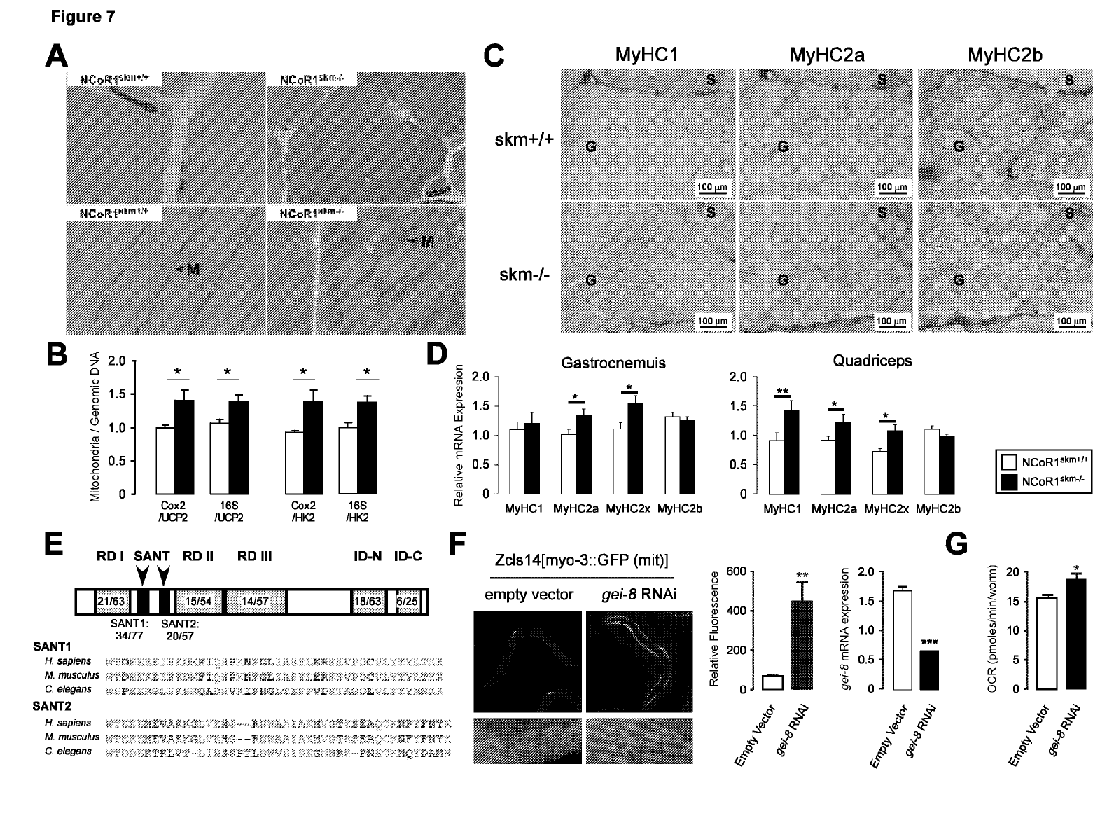

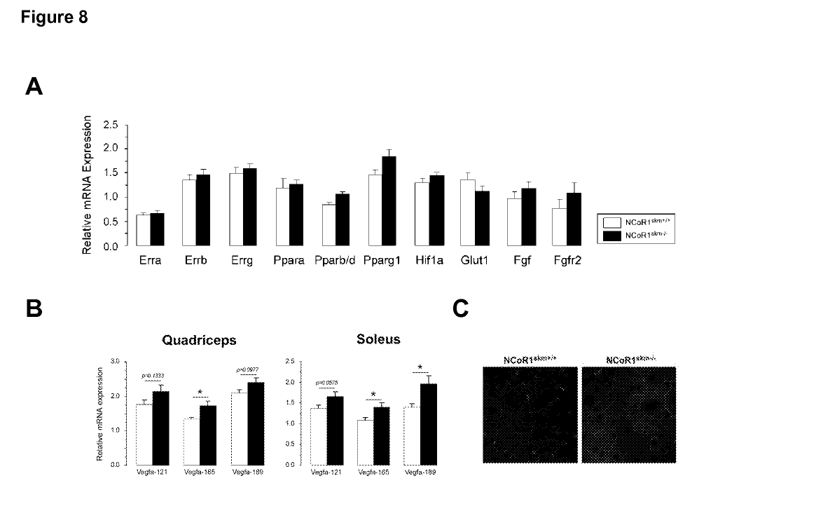

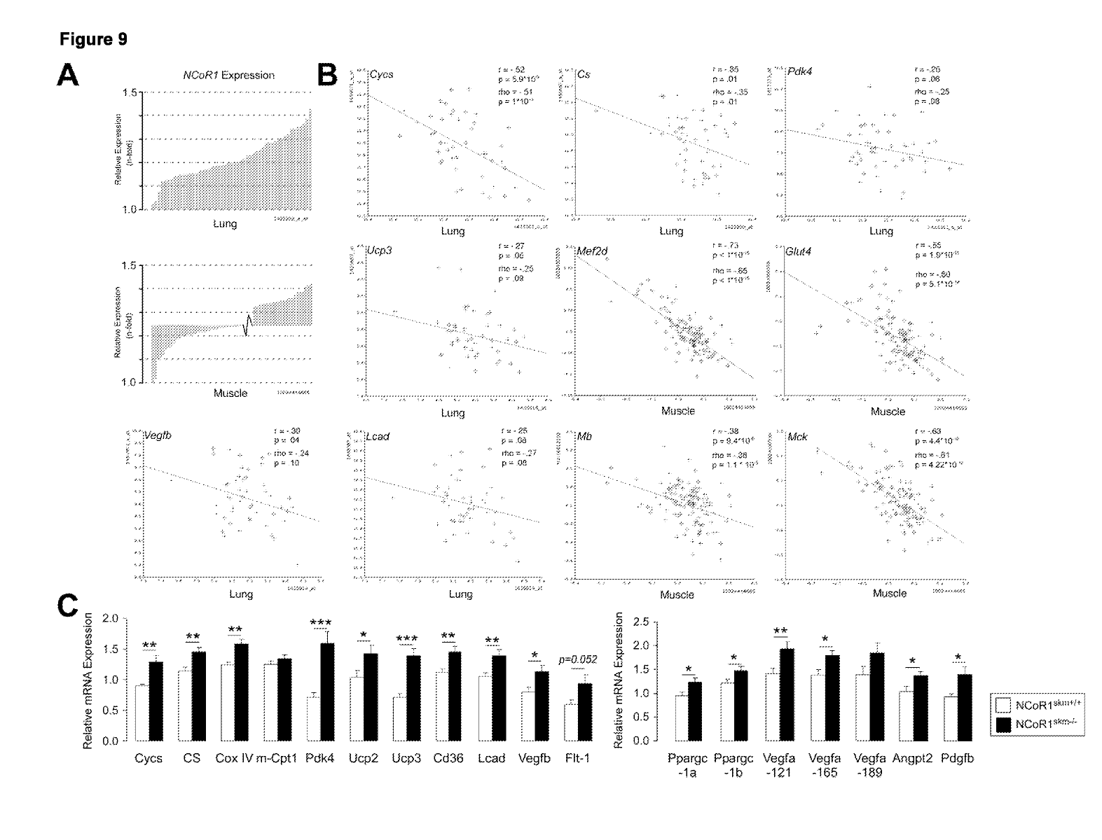

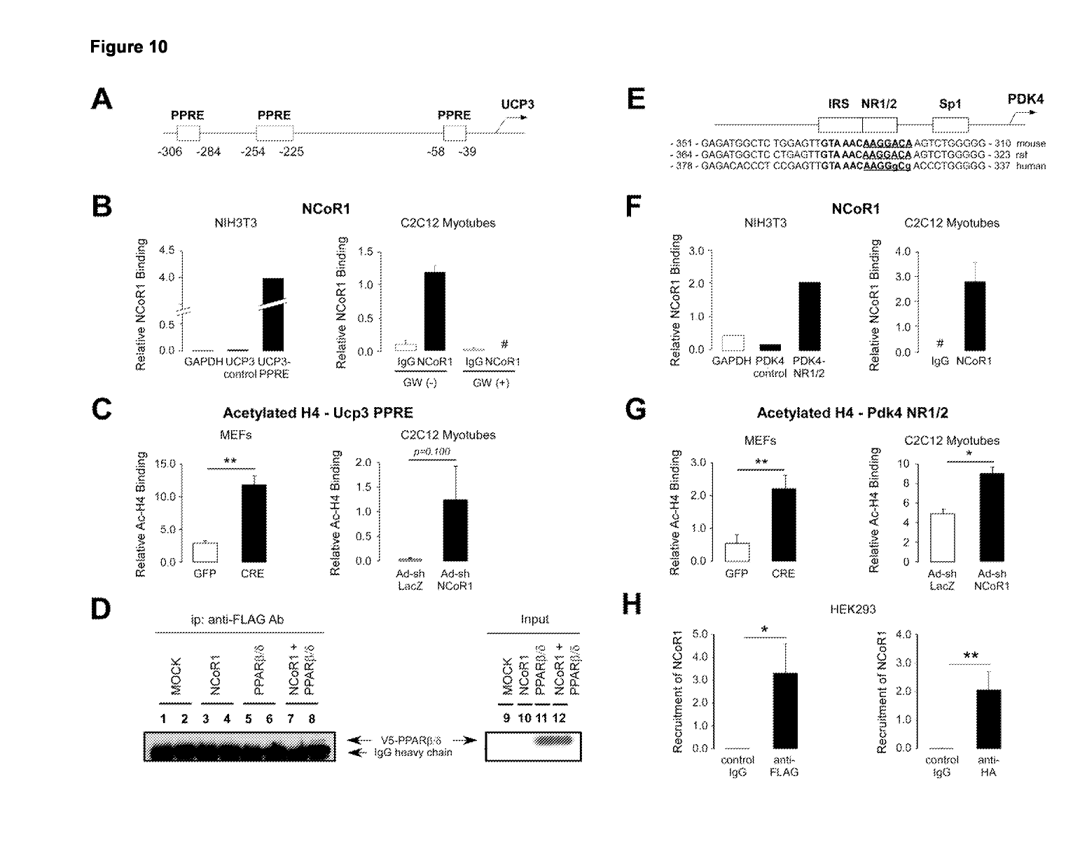

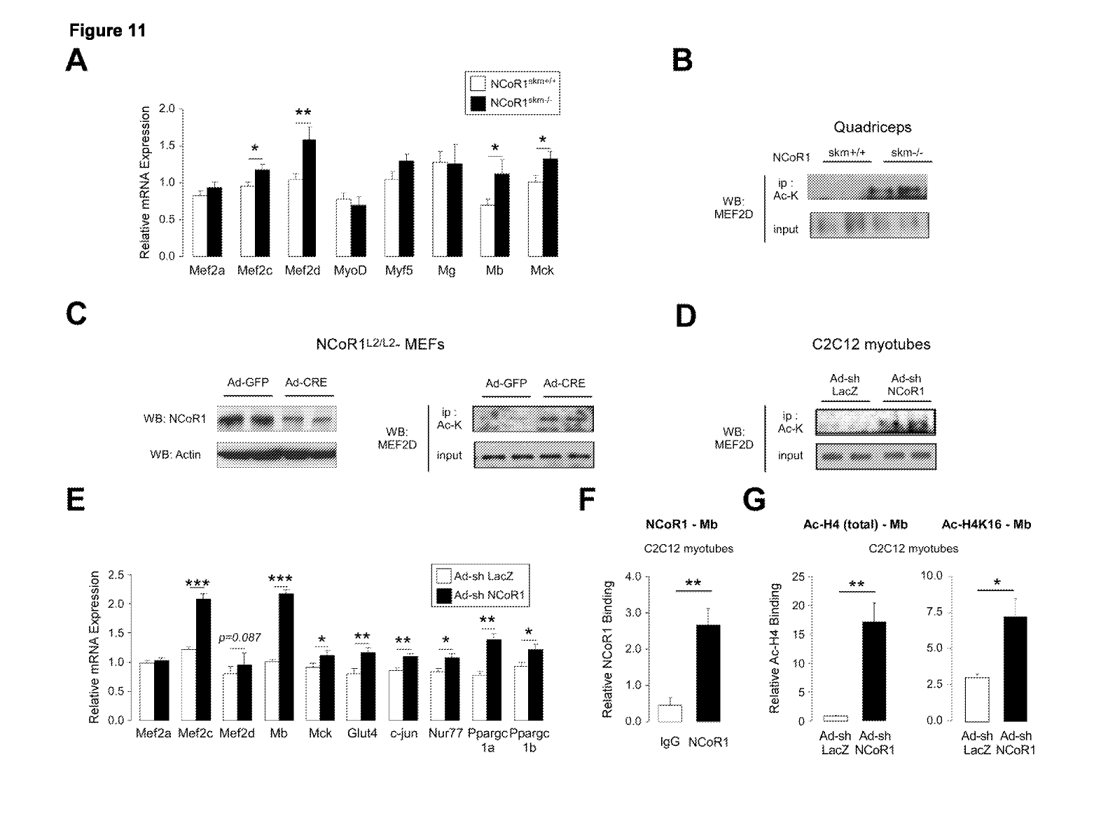

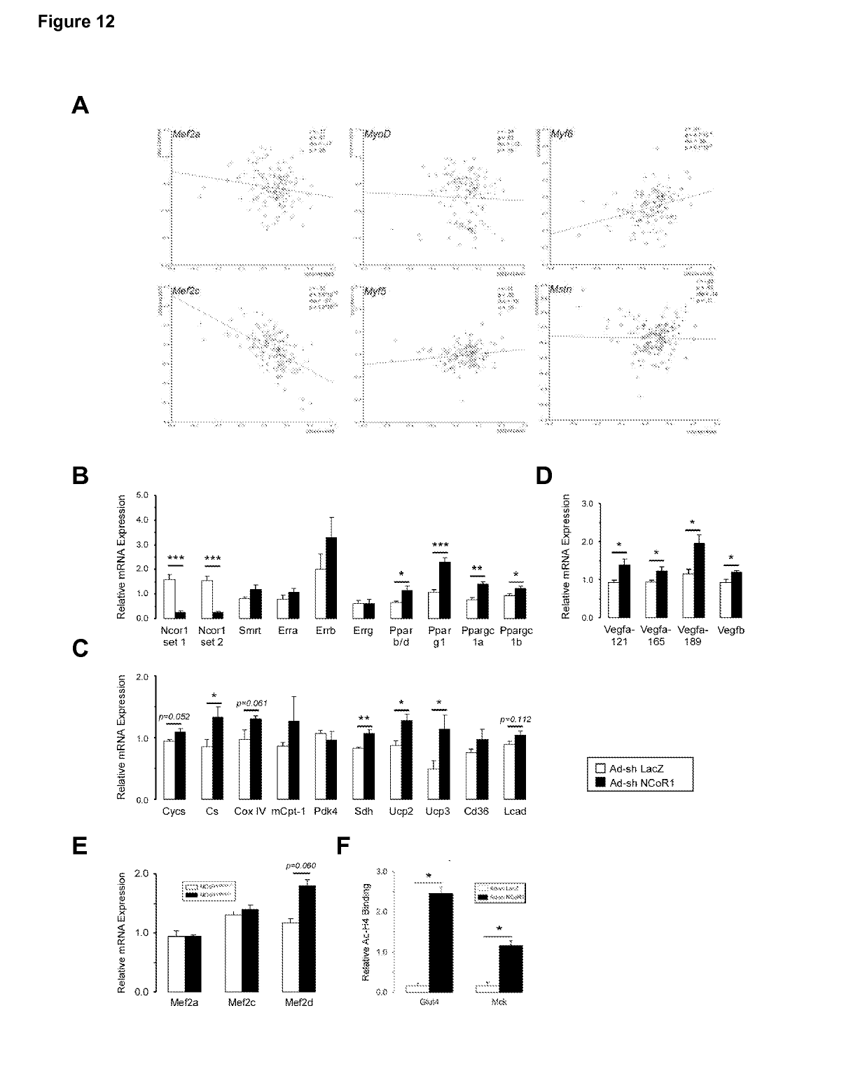

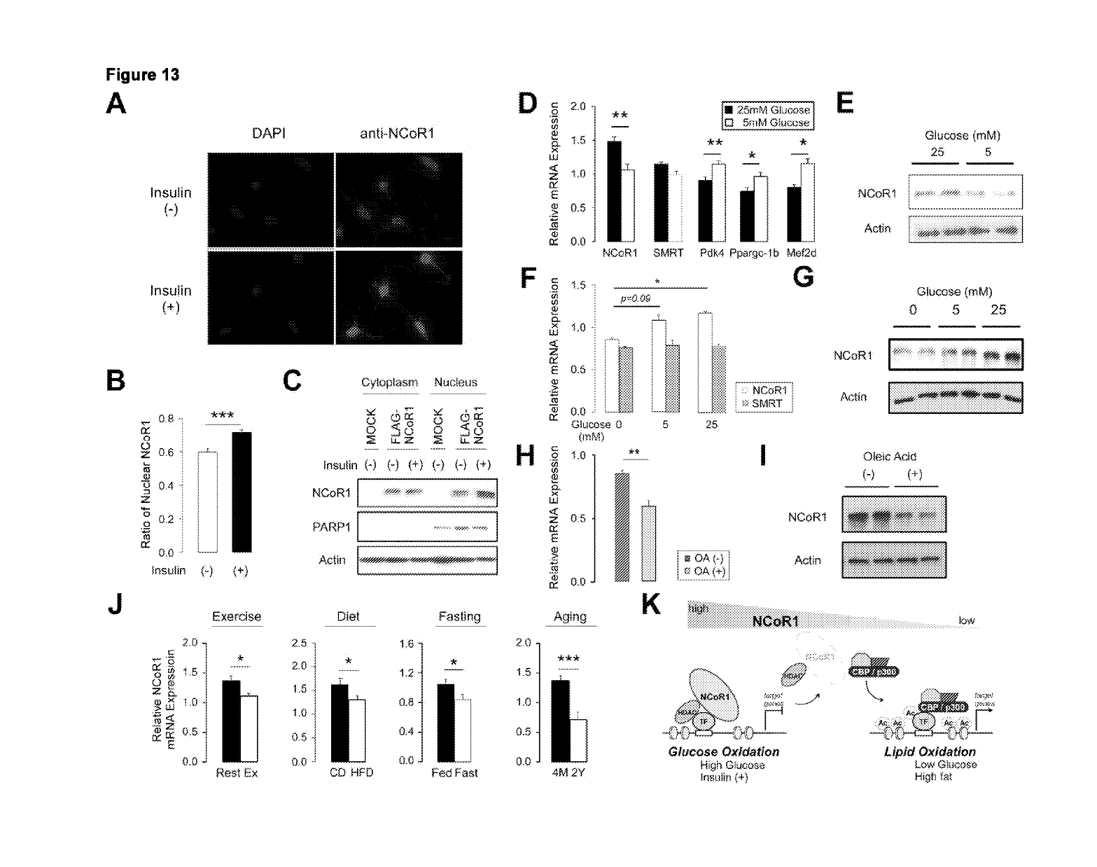

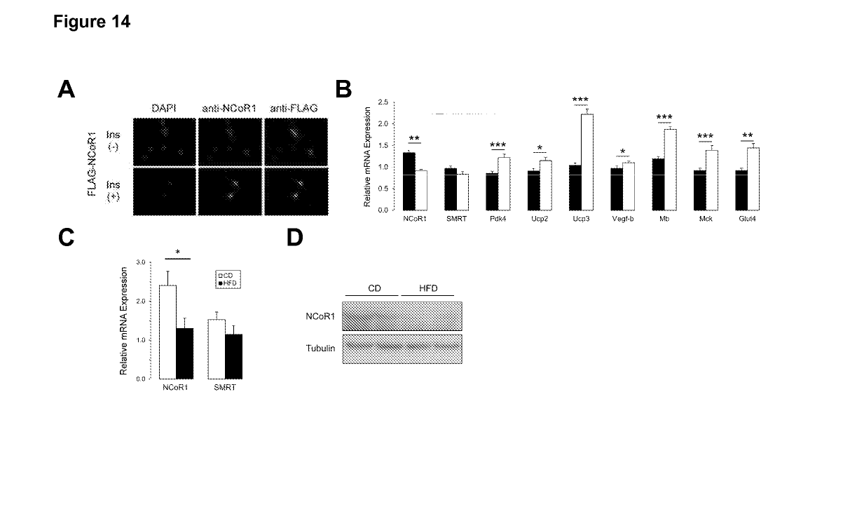

These mice, bearing floxed NCoR1 L2 alleles, were then bred with a skeletal muscle (skm)-specific Cre driver (human α-skeletal actin promoter) (Miniou et al., 1999) to yield NCoR1^{skm-/-} and NCoR1^{skm+/+} mice (FIG. 1). As expected, NCoR1 mRNA expression was significantly decreased in soleus, gastrocnemius and quadriceps and modestly reduced in the heart muscle of NCoR1^{skm-/-} mice, but not altered in other tissues (FIG. 2A). No compensatory induction of the related co-repressor SMRT/NCoR2 (Chen and Evans, 1995) was observed (FIG. 2A). We also tried to determine NCoR1 protein levels in muscle, but failed to detect the endogenous protein with the currently available NCoR1 antibodies.

NCoR1^{skm-/-} mice were indistinguishable from NCoR1^{skm+/+} mice upon visual inspection and no gross organ anomalies were revealed upon autopsy. The relative mass of the soleus muscle was higher, whereas the mass of the gastrocnemius showed a trend towards an increase, which did not reach statistical significance (3A-B). The soleus was also more intensely red and there were larger sections with reddish color in the gastrocnemius in NCoR1^{skm-/-} mice (FIG. 3C). Body weight evolution and food intake of male NCoR1^{skm+/+} and NCoR1^{skm+/+} mice after weaning was comparable both on chow diet (CD) and on high fat diet (HFD) (FIG. 3A). On CD, carbohydrate and lipid profiles were similar, except for LDL cholesterol, which was reduced in NCoR1^{skm-/-} mice (FIG. 2B). In addition to the lower LDL cholesterol on CD, total and HDL cholesterol levels were also reduced in NCoR1^{skm-/-} mice on HFD (FIG. 2B). Furthermore, glucose edged down (p=0.074) in the wake of similar insulin levels on HFD. The slightly reduced area under the curve in intraperitoneal glucose tolerance test (IPGTT; FIG. 4D) and the delayed recovery from hypoglycemia during intraperitoneal insulin tolerance test (IPITT; FIG. 3E) in mutant mice on HFD, may suggest a discrete improvement in insulin sensitivity but without a clear impact on glucose tolerance.

### Example 3

### Enhanced Exercise Performance in NCoR1^{SKM-/-} Mice

We next evaluated energy expenditure by indirect calorimetry and actiometry in CD and HFD fed mice (FIG. 2C and FIG. 3F). Total locomotor activity was significantly higher in NCoR1^{skm-/-} mice. Consistent with this, O₂ consumption (VO₂) was increased under both CD and HFD. Interestingly, the NCoR1^{skm-/-} mice displayed a marked decrease in the respiratory exchange ratio (RER) on a HFD (FIG. 2C), indicating an enhanced use of fat as main energy source. NCoR1^{skm-/-} mice were also more cold tolerant, as they maintained their body temperature better when exposed to 4° C. (FIG. 2D).

Exercise performance was strikingly improved in NCoR1^{skm-/-} mice (FIG. 2E-F and FIG. 5A-D). In endurance exercise, NCoR1^{skm-/-} mice ran for a significantly longer time and distance before exhaustion (FIG. 2E and FIG. 5A-B). The increase of the VO₂ values (ΔVO₂) during exercise and the maximal ability to utilize oxygen during exercise (VO₂.), which critically determines the endurance performance of skeletal muscle, was slightly higher in NCoR1^{skm-/-} mice on both CD (FIG. 5D) and HFD (FIG. 2F). Despite the moderate reduction in NCoR1 mRNA levels in cardiac muscle of NCoR1^{skm-/-} mice, heart rate, blood pressure, cardiac morphology and function were not changed (FIG. 5E-G and Table 3).

TABLE 3
Cardiac functions are not affected in NCoR1^{skm-/-} mice.

| | | skm+/+ | skm−/− |
|---|---|---|---|
| LV | IVSd (mm) | 1.10 ± 0.09 | 0.87 ± 0.06 |
| | IVSs (mm) | 1.54 ± 0.11 | 1.36 ± 0.08 |
| | Dd (mm) | 3.50 ± 0.12 | 3.69 ± 0.08 |
| | Ds (mm) | 2.28 ± 0.16 | 2.40 ± 0.12 |
| | PWd (mm) | 1.02 ± 0.12 | 1.08 ± 0.07 |
| | PWs (mm) | 1.51 ± 0.15 | 1.49 ± 0.07 |
| | mass (mg) | 115.0 ± 12.2 | 108.5 ± 1.8 |
| | volume-d (□l) | 51.5 ± 3.8 | 57.9 ± 3.0 |
| | volume-s (□l) | 18.5 ± 3.3 | 20.6 ± 2.6 |

| | | |
|---|---|---|
| EF (%) | 64.9 ± 4.4 | 64.7 ± 3.4 |
| FS (%) | 35.3 ± 3.0 | 35.0 ± 2.5 |
| CO (ml/min) | 18.3 ± 1.1 | 15.3 ± 1.3 |

n.s. for all parameters
Parameters of cardiac function of NCoR1^{skm+/+} and ^{skm-/-} mice obtained by echocardiography (n = 8).
LV = left ventricle.
IVS = interventricular septum thickness.
Dd = end-diastolic diameter.
Ds = end-systolic diameter.
PW = Postelo-lateral wall thickness.
EF = ejection fraction.
FS = fractional shortening.
CO = cardiac output.
D = diastolic.
S = systolic.

### Example 4

### NCoR1^{SKM-/-} Muscle Demonstrates Increased Oxidative Capacity

The enhanced exercise capacity, associated with the increase in overall muscle mass and change in muscle appearance, led us to examine muscle morphology. Upon staining muscles with hematoxylinieosin or toluidine blue, not only the diameter of single muscle fibers was larger, but also the connective tissue between the muscle bundles was less abundant in NCoR1^{skm-/-} mice (FIG. 4A-B and FIG. 6A). The increased number of intensely stained fibers upon succinate dehydrogenase (SDH) and cytochrome oxidase (COX) (FIG. 4C-D) staining, further testified of increased mitochondrial activity in the NCoR1^{skm-/-} gastrocnemius. Two mitochondrial DNA markers, cyclooxygenase 2 (Cox2) and 16S ribosomal RNA, normalized by genomic DNA markers [uncoupling protein 2 (Ucp2) and hexokinase 2 (Hk2)] were both significantly higher in NCoR1^{skm-/-} muscle, indicative of increased mitochondrial content (FIG. 7B). This observation was also underscored by electron microscopy, which revealed more abundant and larger mitochondria with normal structure (FIG. 7A). Immunohistochemical analysis of the myosin heavy chain (MyHC) isoforms (Schiaffino et al., 1989) demonstrated a decreased number of the more glycolytic MyHC2b fibers, with a concomitant increase in the number of more oxidative MyHC2x and 2a fibers in the NCoR1^{skm-/-} gastrocnemius (FIG. 7C). This observation was consolidated by analysis of MyHC isoform mRNAs, which indicated an increased expression of the mRNAs of MyHC2x and 2a (more oxidative fibers) compared to that of MyHC2b (more glycolytic) in both NCoR1^{skm-/-} gastrocnemius and quadriceps (FIG. 7D). In quadriceps, but not gastrocnemius, the expression of MyHC1 mRNA was also increased. Finally, staining of platelet-endothelial cell adhesion molecule (PECAM)-1, an endothelial cell marker of angiogenesis and tissue vascularization, which contributes to enhanced myocellular aerobic capacity, also increased in NCoR1^{skm-/-} muscle (FIG. 8C).

### Example 5

### The Control of Muscle Mitochondria by NCoR1 is Conserved in C. elegans

To investigate whether the effects of NCoR1 deficiency are evolutionary conserved, we took advantage of the power of C. elegans genetics. A protein blast search indicated that gei-8 (GEX interacting protein family member 8) is the only putative NCoR1 homolog in the C. elegans genome. Further analysis showed that the total amino acid sequence of gei-8 is 43% homologous to mouse NCoR1 and contained conserved SANT (switching-defective protein 3 (Swi3), adaptor 2 (Ada2), nuclear receptor co-repressor (NCoR), transcription factor (TF)IIIB)) domains (34% identical/77% similar for SANT1; 20% identical/57% similar for SANT2) (FIG. 7E). Other important functional domains (Repressor Domain (RD) and nuclear receptor Interaction Domain (ID)) were also conserved (FIG. 7E). Upon the robust gei-8 knockdown in worms expressing a mitochondrial GFP-reporter driven by the muscle-specific myo-3 promoter, a striking enlargement of the mitochondria was observed in body wall muscle (FIG. 7F). This result is not due to an indirect effect on transcriptional activity through the myo-3 promoter because no increase in GFP expression is observed with another strain carrying the ₚmyo-3::GFP reporter (FIG. 6B). We also measured O₂ consumption in NR350 transgenic worms fed with gei-8 dsRNA. NR350 worms lack rde-1, an essential component of the RNAi machinery encoding a member of the PIWI/STING/Argonaute family, in all tissues except the body wall muscle in which the wild-type rde-1 gene has been rescued using the hlh-1 promoter (Durieux et al., 2011). Consistent with the effects observed in the mouse, also the muscle-specific knockdown of gei-8 enhanced O₂ consumption in these NR350 worms (FIG. 7G), suggesting that the function of gei-8 to control mitochondrial metabolism is conserved through evolution.

### Example 6

### NCoR1 Negatively Correlates with Key Mitochondrial and Myogenic Genes

After establishing these striking mitochondrial effects of NCoR1 in mice and worms, we exploited a complementary systems genetics approach to evaluate NCoR1s molecular coexpression partners in the mouse (Argmann et al., 2005; Houtkooper et al., 2010). Expression of NCoR1 in two panels of genetically heterogeneous mice, made by intercrossing C57BL/6 with C3H/HeJ (the BXH F2 cross), or C57BL/6 with DBA/2J (the BXD genetic reference population) mice, varied ±1.5-fold between cases in both lung and muscle (FIG. 9A). A large number of transcripts covaried significantly with NCoR1 in the different mice lines belonging either to the BXH cross or BXD strains. Most distinctively, only a fraction of these covariates were negative, which was against the dogma expected for a corepressor such as NCoR1s. In skeletal muscle from the BXH cross (n=124 females), strong covariates of NCoR1 include myocyte-specific enhancer factor 2D (Mef2d), myoglobin (Mb), muscle creatine kinase (Mck), and glucose transporter type 4 (Glut4) (van Nas et al., 2010). A similar analysis of lung tissue from the BXD cross (n=51 strains) includes genes such as cytochrome c (Cycs), citrate synthase (Cs), pyruvate dehydrogenase kinase 4 (Pdk4), uncoupling protein 3 (Ucp3), vascular endothelial growth factor b (Vegfb), and long chain acyl-CoA dehydrogenase (Lcad) (FIG. 9B). This analysis significantly extends the number of NCoR1 targets and covariates, with several of them being consistent with increased mass and mitochondrial biogenesis observed in NCoR1^{skm-/-} muscle.

This initial set of NCoR1 covariates (FIG. 9B) was then included together with other potential candidates for qRT-PCR analysis in mixed fiber muscle, including the gastrocnemius and quadriceps (FIG. 9C and FIG. 8A). Whereas the mRNAs of most relevant NRs were unchanged, mRNA levels of PGC-1α and β (Ppargc1a and 1b) increased. Several genes involved in mitochondrial function, including those encoding for proteins involved in TCA cycle and oxidative phosphorylation [Cs, cytochrome c oxidase subunit IV (CoxIV), Pdk4], uncoupling [Ucp2 and Ucp3], fatty acid uptake and metabolism [Cd36 and Lead], were robustly induced in NCoR1^{skm-/-} muscle. In addition, mRNA levels of Vegfb and its receptor Flt1, which regulates trans-endothelial fatty acid transport (Hagberg et al., 2010), were also induced. Interestingly, the expression of hypoxia inducible factor (Hif) 1α and of its targets, glucose transporter 1 (Glut1), fibroblast growth factor (Fgf) and Fgf-receptor 2 (Fgfr2), were unchanged (FIG. 8A), whereas all three Vegfa isoforms, i.e. Vegfa-121, -165, and -189, were induced in NCoR1^{-/-} quadriceps, gastrocnemius, and soleus (FIG. 9C and FIG. 8B). Together with this increase in Vegfa, both Angpt2 and Pdgfb mRNA levels were induced (FIG. 9C), suggesting that myocellular aerobic capacity is facilitated by a HIF1α-independent angiogenic pathway in NCoR1^{skm-/-} mice (Arany et al., 2008).

### Example 7

### Enhanced PPARIβ/δ and/or ERR Function in NCoR1^{SKM-/-} Muscle

Several genes whose expression is changed in the absence of NCoR1 are PPARβ/δ and/or ERR targets (FIG. 9). Since the expression of PPARβ/δ and/or ERR was unchanged in NCoR1^{skm-/-} mice (FIG. 8A), a direct effect of NCoR1 on the expression of these targets through the activation of these NRs was expected. As cases in point to demonstrate the recruitment of NCoR1 to these genes, we selected the mouse Ucp3 and Pdk4 promoters, which contain three PPAR responsive elements (PPREs) (FIG. 10A) and extended NR half-sites (NR1/2), known to bind members of the ERR subfamily (FIG. 10E) (Zhang et al., 2006), respectively. We first used NIH-3T3 cells in which an epitope-tagged version of NCoR1 (NCoR1-FLAG) was expressed. The two PPREs adjacent to the Ucp3 transcription start site recruited NCoR1 more efficiently, compared to two control sequences in the Gapdh and Ucp3 promoter that lack PPREs (FIG. 10B, left). Likewise, NCoR1 bound avidly to the mouse Pdk4 promoter NR1/2 site in transfected NIH-3T3 cells (FIG. 10F, left). Although there is a two nucleotide difference in NR1/2 site of the human Pdk4 promoter (FIG. 10E), NCoR1 and ERRα were also recruited to this site in human HEK293 cells (FIG. 10H).

We then used a highly specific NCoR1 antibody, which we recently generated, for ChIP experiments in C2C12 myotubes. Confirming our data in NIH-3T3 cells that express NCoR1-FLAG, endogenous NCoR1 occupied the same Ucp3 PPREs (FIG. 10B, right). The recruitment of NCoR1 to the Ucp3 promoter was robustly inhibited by the addition of the selective PPARβ/δ ligand GW501516. Likewise, endogenous NCoR1 was readily detected on the NR1/2 in the Pdk4 promoter in C2C12 myotubes (FIG. 10F, right).

Subsequently, we explored whether NCoR1 gene deletion in NCoR1^{L2/L2}MEFs by means of adenoviral Cre recombination, or NCoR1 gene knockdown in C2C12 myotubes infected by an NCoR1 shRNA adenovirus, modulates histone H4 acetylation on the Ucp3 and Pdk4 promoters. Consistent with NCoR1 binding to these promoters in NIH-3T3 cells and C2C12 myotubes (FIGS. 10B and F), NCoR1 deletion or silencing induced H4 acetylation of both target promoters in MEFs and C2C12 cells, indicating chromatin opening (FIGS. 10C and G).

To further consolidate these observations, we analyzed whether NCoR1 interacts directly with PPARβ/δ or ERRα, using nuclear extracts of HEK293 cells, transfected with tagged versions of NCoR1, PPARβ/δ or ERRα. Although a specific association between NCoR1 and PPARβ/δ was evident in these co-IP experiments (FIG. 10D, lane 8), we failed to detect a similar interaction between ERRα and NCoR1.

### Example 8

### MEF2 is Hyperacetylated and Activated in the Absence of NCoR1

The increased muscle mass observed in NCoR1^{skm-/-} mice indicated that the absence of NCoR1 not only induced oxidative metabolism, but also stimulated myogenesis. In line with this, mRNA levels of two markers of myogenesis, Mb and Mck were increased in NCoR1^{skm-/-} quadriceps (FIG. 11A). Amongst several myogenic regulatory factors, only the expression of two Mef2 family members, i.e. Mef2c and Mef2d, negatively correlated with NCoR1 expression in our systems genetics analysis (FIG. 9 and FIG. 12A). The selective induction of Mef2c and Mef2d mRNA was furthermore confirmed by qRT-PCR of NCoR1^{skm-/-} gastrocnemius and quadriceps, while no changes were found in MyoD, myf5, and myogenin mRNA (FIG. 11A and FIG. 12E).

The activity of MEF2 family members is not only controlled by their expression levels, but is also modulated by their acetylation status. MEF2 is acetylated and activated by p300, whereas it is deacetylated by HDAC3 and HDAC4, which are part of the NCoR1 corepressor complex (Ma et al., 2005; Nebbioso et al., 2009). Since the expression of the Mef2d isoform is most prominently correlated with NCoR1 expression, we investigated MEF2D acetylation in gastrocnemius and found that its acetylation levels were enhanced in NCoR1^{skm-/-} mice (FIG. 11A-B).

We then compared the acetylation of MEF2D in floxed NCoR1^{L2/L2}MEFs, infected with an adenovirus expressing either GFP as control or Cre-recombinase to reduce NCoR1 protein expression (FIG. 11C, left panel). Whereas MEF2D protein levels were stable in NCoR1^{-/-} MEFs, perhaps due to the more acute nature of the deletion, MEF2D was robustly hyperacetylated when NCoR1 levels were attenuated (FIG. 11C, right panel). Likewise, a slight but consistent MEF2D hyperacetylation was observed in C2C12 myotubes infected with Ad-shNCoR1 to knockdown NCoR1 expression (FIG. 11D and FIG. 12B-D), further underscoring the importance of MEF2D deacetylation by the NCoR1 complex.

Given the induction of MEF2D expression and its hyperacetylation and consistent with our systems genetics analysis (FIG. 9B), mRNA levels of the MEF2 targets, Mb and Mck, were robustly induced in NCoR1^{skm-/-} gastrocnemius (FIG. 11A). Silencing of NCoR1 in C2C12 myotubes also resulted in a similar induction of several MEF2 target genes, including Mb, Mck, Glut4, c-jun, Nur77, PGC-1α, PGC-1β (FIG. 11E and FIG. 12B-D). In line with these data, endogenous NCoR1 was readily detected on MEF2 binding sites on these target promoters in C2C12 myotubes, as illustrated for the Mb promoter (FIG. 11F). The induction of these MEF2 targets by NCoR1 knock-down was furthermore accompanied by H4K16 and global H4 hyperacetylation on their promoters (e.g. Mb, Glut4, Mck) (FIG. 11G and FIG. 12F).

### Example 9

### NCoR1 Levels are Regulated in Response to Physiological Stimuli

We next investigated whether NCoR1 function could be altered by different physiological stimuli in vitro and in vivo. One hour after stimulation of 293T cells with 1 μM insulin, higher amounts of endogenous NCoR1 (FIG. 13A) or transfected FLAG-NCoR1 (FIG. 14A) were detected in nuclei as evidenced by immunofluorescence and subcellular fractionation (FIG. 13A-C).

At the transcriptional level, NCoR1 mRNA changed in response to different concentrations of glucose in the culture media (FIG. 13D-E). Growing MEFs in low glucose decreased NCoR1 mRNA (FIG. 13D) and protein (FIG. 13E) levels, concomitant with the induction of its target genes (Pdk4, Vgefb, Mef2d, etc.). Similar results, i.e. decreased mRNA levels of NCoR1 associated with increased expression of its targets (Pdk4, Ucp2, Ucp3, Vegfb, Mb, Mck, Glut4), were also obtained in glucose-deprived C2C12 myotubes (FIG. 14B). Interestingly, the reduction of glucose decreased mRNA levels of NCoR1, but not those of SMRT (FIG. 13F). The tight dose-dependent correlation between NCoR1 expression and glucose levels in the culture medium (FIG. 13E-F), suggested the possibility that NCoR1 could block the oxidation of lipid substrates when glucose was available. We therefore evaluated NCoR1 mRNA and protein in MEFs cultured in different fatty acid concentrations (FIG. 13H-I). The addition of oleic acid (OA) to the medium to force fatty acid oxidation also decreased NCoR1 levels, independently of the glucose concentration (FIG. 13H). These effects seemed again specific to NCoR1, as only a small difference in SMRT mRNA levels was observed with OA in the absence of glucose. Together, these data indicate that settings that favor fatty acid oxidation (i.e. low glucose, low insulin, and high fatty acid) are all associated with a reduction of NCoR1.

We then test whether different conditions that enhance fatty acid oxidation also modulate NCoR1 mRNA levels in the muscle in vivo. This was indeed the case, as muscle NCoR1, but not SMRT, mRNA levels decreased after exercise (3 hrs after a resistance run), high fat feeding (20 wks of high fat feeding), fasting (after a 16 hr fast), and aging (6-mo vs 2-yr-old mice) (FIG. 13J). Interestingly, the reduction in NCoR1 mRNA levels match well with the potentiation of lipid oxidation after exercise (Kiens and Richter, 1998; Pilegaard et al., 2000), high fat feeding (Watanabe et al., 2006), and in fasting mice (Storlien et al., 2004). Also in epidydimal white adipose tissue, HFD feeding reduced specifically NCoR1, and not SMRT, mRNA (FIG. 14C). Unlike for the muscle, where we were unable to detect NCoR1 with the available antibodies, NCoR1 protein levels almost disappeared from epidydimal fat upon HFD (FIG. 14D). Altogether, these data led us to suggest that NCoR1 is a negative transcriptional regulator of fatty acid oxidation and that a reduction of NCoR1 enables the muscle (and adipose tissue), to deal with lipid substrates more efficiently.

1. A method of increasing muscle mass or muscle mitochondrial oxidative metabolism comprising administering to subject in need thereof one or more compounds that decrease muscle-specific nuclear receptor corepressor 1 (NCoR1) expression or activity.

2. A method of increasing mitochondrial number and function in a cell comprising contacting a cell with one or more compounds that decrease muscle-specific nuclear receptor corepressor 1 (NCoR1) expression or activity.

3. A method of treating a disorder associated with mitochondrial dysfunction or a muscular dystrophy disorder comprising administering to a subject in need thereof one or more compounds that decrease muscle-specific nuclear receptor corepressor 1 (NCoR1) expression or activity.

4. The method of claim 3, wherein the disorder associated with mitochondrial dysfunction is a genetic mitochondrial disease or a metabolic disorder.

5. The method of claim 4, wherein the metabolic disorder is type II diabetes or obesity.

6. The method of claim 3, wherein the muscular dystrophy disorder is an inherited muscular dystrophy disorder or an acquired muscular dystrophy disorder.

7. The method of claim 1, wherein the compound is a NCoR1 antibody or a nucleic acid that inhibits NCoR1 expression or activity.

8. The method of claim 1, wherein the compound inhibits or dissociates a NCoR1-histone deacetylases (HDAC) complex.

9. The method of claim 8 wherein the compound is an HDAC inhibitor.

10. The method of claim 7, wherein the nucleic acid is a nucleic acid that is complementary to a NCoR1 nucleic acid or fragment thereof.

11. The method of claim 2, wherein the cell is a muscle cell or an adipocyte.
